(19) United States
(12) Patent Application Publication
(10) Pub. No.: US 2023/0172198 A1
(43) Pub. Date: Jun. 8, 2023

(54) ANTI-PATHOGEN STRUCTURE, METHOD FOR PRODUCING ANTI-PATHOGEN STRUCTURE, APPARATUS FOR PRODUCING ANTI-PATHOGEN STRUCTURE, AND LIQUID COMPOSITION

(71) Applicants: Ryo Kawamura, Kanagawa (JP); Satoshi Yamamoto, Kanagawa (JP); Keigo Takauji, Kanagawa (JP); Nana Akiba, Kanagawa (JP); Yuki Hoshikawa, kanagawa (JP); Miku Ohkimoto, Kanagawa (JP); Yohei Shiren, Tokyo (JP); Tatsuru Moritani, Kanagawa (JP); Naoki Shiraishi, Kanagawa (JP); Yuichi Sato, Kanagawa (JP)

(72) Inventors: Ryo Kawamura, Kanagawa (JP); Satoshi Yamamoto, Kanagawa (JP); Keigo Takauji, Kanagawa (JP); Nana Akiba, Kanagawa (JP); Yuki Hoshikawa, kanagawa (JP); Miku Ohkimoto, Kanagawa (JP); Yohei Shiren, Tokyo (JP); Tatsuru Moritani, Kanagawa (JP); Naoki Shiraishi, Kanagawa (JP); Yuichi Sato, Kanagawa (JP)

(73) Assignee: Ricoh Company Ltd., Tokyo (JP)

(21) Appl. No.: 17/905,709

(22) PCT Filed: Feb. 10, 2021
(86) PCT No.: PCT/JP2021/004896

§ 371 Date: Sep. 6, 2022

(30) Foreign Application Priority Data
Mar. 13, 2020 (JP) 2020-044218
Nov. 24, 2020 (JP) 2020-194006

(51) Int. Cl. A01N 25/34; A61L 2/235; C08J 9/14; A01P 1/00

(52) U.S. Cl. CPC A01N 25/34; A61L 2/235; C08J 9/142; A01P 1/00; C08J 2335/02; C08J 2203/12; C08J 2205/042

(57) ABSTRACT

An anti-pathogen structure includes a resin structure having a plurality of openings in a surface of the resin structure, wherein the resin structure has an antimicrobial activity or an antiviral activity.

### TECHNICAL FIELD

The present disclosure relates to an anti-pathogen structure, a method for producing an anti-pathogen structure, an apparatus for producing an anti-pathogen structure, and a liquid composition.

### BACKGROUND ART

In recent years, many products exhibiting an antimicrobial activity or an antiviral activity have been commercially available. The antimicrobial activity means, for example, a property of decreasing the number of microorganisms. The antiviral activity means a property of decreasing the number of viruses, or a property of decreasing activity (e.g., an infection ability to a host and a proliferation potency in a host) in the whole viruses. As a method for achieving the antimicrobial activity or the antiviral activity, for example, a method for incorporating, to a structure, a pharmaceutical agent that gives some injure to or kills microorganisms or viruses, and a method where a structure having a special surface structure is used to give some injure to or kill microorganisms or viruses that are in contact with the surface structure have been known.

Non Patent Literature 1 discloses that cicada wings have fine projection structures on the surfaces, and the projection structures exhibit the antimicrobial activity. More specifically, it discloses that the pillars with the fine projection structures (nanopillars) destroy the outer shell parts (e.g., cell membrane and cell wall) of microorganisms to exhibit the antimicrobial activity.

Non Patent Literature 2, Patent Literature 1, and Patent Literature 2 disclose that even when a structure imitating the aforementioned pillars with the fine projection structures (nanopillars) is artificially produced, the antimicrobial activity can be exhibited.

### CITATION LIST

### Patent Literature

PTL 1: Japanese Unexamined Patent Application Publication No. 2019-72475
PTL 2: Japanese Patent No. 6454710

### Non Patent Literature

NPL 1: Elena P. Ivanova et al., “Natural Bactericidal Surfaces: Mechanical Rupture of Pseudomonas aeruginosa Cells by Cicada Wings”, small, 2012, Volume 8, Issue 16, pp. 2489-2494
NPL 2: Elena P. Ivanova et al., “Bactericidal activity of black silicon”, Nature Communications, Nov. 26, 2013

### SUMMARY OF INVENTION

### Technical Problem

However, the conventional anti-pathogen structures have a problem that the antimicrobial activity or the antiviral activity easily decreases.

### Solution to Problem

According to one aspect of the present disclosure, an anti-pathogen structure includes a resin structure having a plurality of openings in a surface of the resin structure. The resin structure has an antimicrobial activity or an antiviral activity.

### Advantageous Effects of Invention

The present disclosure achieves an excellent effect of preventing an antimicrobial activity or an antiviral activity being decreased.

### BRIEF DESCRIPTION OF DRAWINGS

FIG. 1 is a schematic view presenting one example of an apparatus for producing an anti-pathogen structure in order to achieve a method for producing an anti-pathogen structure of an embodiment of the present disclosure.

FIG. 2 is a view obtained when a surface of a resin structure (anti-pathogen structure), which includes: skeletons having such a shape that a plurality of particles are coupled to each other; and openings shaped by the skeletons, is observed with a scanning electron microscope (SEM).

FIG. 3 is a view obtained when a surface of a resin structure (anti-pathogen structure), which includes skeletons having a substantially flat shape; and openings shaped by the skeletons, is observed with a scanning electron microscope (SEM).

FIG. 4 is a view obtained when a surface of an anti-pathogen structure of Example 1 is observed with a scanning electron microscope (SEM).

FIG. 5 is a view obtained when a surface of a structure of Comparative Example 1 is observed with a scanning electron microscope (SEM).

FIG. 6 is a view obtained when a surface of a structure of Example 4 is observed with a scanning electron microscope (SEM).

### DESCRIPTION OF EMBODIMENTS

Hereinafter, one embodiment of the present disclosure will be described.

<<Anti-Pathogen Structure>>

An anti-pathogen structure of the present embodiment includes a resin structure having a plurality of openings in a surface of the resin structure, and further may include other substances if necessary. The anti-pathogen structure may not include other substances and may include only the resin structure.

The anti-pathogen structure represents a concept including an antimicrobial structure exhibiting an antimicrobial activity and an antiviral structure exhibiting an antiviral activity. The anti-pathogen structure is a structure exhibiting the antimicrobial activity or the antiviral activity as a whole because a resin structure constituting the anti-pathogen structure has the antimicrobial activity or the antiviral activity. In other words, the resin structure per se is a structure that can exhibit the antimicrobial activity or the antiviral activity. Note that, when inclusion of only the resin structure exhibits the antimicrobial activity or the antiviral activity, a pharmaceutical agent having an antimicrobial activity (hereinafter may be referred to as an antimicrobial agent) or a pharmaceutical agent having an antiviral activity (hereinafter may be referred to as an antiviral agent) as other substances may be additionally included in the structure or may be born on the surface of the structure. In the following description, when both the antimicrobial activity and the antiviral activity are collectively referred to, these activities are referred to as an “anti-pathogen activity”. Note that, the pathogen generally represents one that has a property of causing disease in an organism as a host. However, in the present disclosure, the pathogen represents a concept referring collectively to both microorganisms and viruses, regardless of whether they have a property of causing diseases.

The antimicrobial activity refers to a property of decreasing the number of microorganisms by contacting the resin structure with microorganisms to have some effect on the microorganisms (e.g., injury and kill microorganisms). That is, it cannot be said that when it is difficult to contact the resin structure with microorganisms because the resin structure is sealed or tightly sealed with other members, the resin structure has an antimicrobial activity. Here, the phrase “decreasing the number of microorganisms” means that the number of microorganisms applied to a test piece (test piece C) including an anti-pathogen structure is decreased over time compared to the number of microorganisms applied to a test piece (test piece B), where the test piece (test piece B) is formed of the same material as the material constituting the anti-pathogen structure, but has a flat surface structure and does not have a plurality of openings. A method for confirming this property is not particularly limited. Examples of the method include: a method for directly observing movement of microorganisms using, for example, a fluorescence microscope, a method for observing dead bodies of microorganisms using SEM, and a confirmation method using, for example, an antimicrobial test. Specifically, the antimicrobial test is preferably a test performed according to the method described in, for example, JIS Z 2801 (2012), JIS Z 2901 (2018), and ISO 22196 (2011).

When the test is performed according to the method of JIS Z 2801 (2012), the case where the antibacterial activity value evaluated in the present test is 0.3 or more is preferably judged as having an antimicrobial activity. The antibacterial activity value is preferably 0.5 or more, more preferably 1.0 or more, still more preferably 1.5 or more, particularly preferably 2.0 or more. Here, the antibacterial activity value obtained according to the method of JIS Z 2801 (2012) is represented by the following numerical formula. Specifically, the same bacterial culture is each inoculated into an unprocessed test piece (test piece A) as a glass substrate, a test piece B formed on the test piece A, and a test piece C formed on the test piece A. Then, the viable cell count obtained after 24 hours is measured, and an antibacterial activity value is calculated based on the following numerical formula. The case where the antibacterial activity value is 2 or more may be defined as an antimicrobial material. However, in the present embodiment, the case where the antibacterial activity value is 0.3 or more is judged as “having an antimicrobial activity” in terms of preventing proliferation of microorganisms.

Antibacterial activity value=(log B−log A)−(log C−log A)
A: An average value of viable cell counts on test piece A obtained after 24 hours.
B: An average value of viable cell counts on test piece B obtained after 24 hours.
C: An average value of viable cell counts on test piece C obtained after 24 hours.
   When the test is performed according to the method of ISO 22196 (2011), the case where the antibacterial activity value evaluated in the present test is 0.3 or more is preferably judged as having an antimicrobial activity. The antibacterial activity value is preferably 0.5 or more, more preferably 1.0 or more, still more preferably 1.5 or more, particularly preferably 2.0 or more. Here, the antibacterial activity value obtained according to the method of ISO 22196 (2011) is represented by the following numerical formula. Specifically, the same bacterial culture is each inoculated into a test piece B and a test piece C, and the viable cell count obtained after 24 hours is measured, to calculate the antibacterial activity value based on the following numerical formula. Note that, JIS Z 2801 (2012) and ISO 22196 (2011) are standards that substantially correspond to each other.

Antibacterial activity value=Ut−At
Ut: An average value of common logarithm values of viable cell counts on test piece B obtained after 24 hours
At: An average value of common logarithm values of viable cell counts on test piece C obtained after 24 hours

The antiviral activity refers to a property of decreasing the number of viruses or a property of decreasing activity (e.g., an infection ability to a host and a proliferation potency in a host) in the whole viruses, by contacting the resin structure with viruses to have some effect on the viruses (e.g., injury and kill viruses). That is, it cannot be said that when it is difficult to contact the resin structure with viruses because the resin structure is sealed or tightly sealed with other members, the resin structure has an antiviral activity. Here, the phrase “decreasing the number of viruses” or “decreasing activity in the whole viruses” means that the number of viruses applied to a test piece (test piece X) including an anti-pathogen structure or an activity obtained in the whole viruses is decreased over time compared to the number of viruses or an activity obtained in the whole viruses that is applied to a test piece (test piece Y), where the test piece (test piece Y) is formed of the same material as the material constituting the anti-pathogen structure, but has a flat surface structure and does not have a plurality of openings. A method for confirming the aforementioned properties is not particularly limited. For example, the following method is used. Specifically, viruses having the same concentration are each applied to a test piece X and a test piece Y, and are left to stand for a certain period of time. Then, the viruses that have left to stand are each exposed to a host, to infect the host with the viruses. Then, whether the host is infected with the viruses and whether the host lives or dies are observed. When the onset is not observed or when the host does not die, a partial tissue of the host obtained after a certain period of time has passed since the exposure of the viruses is removed, pulverized, and suspended to prepare a suspension. Then, dilution series of the suspension are prepared, and the dilution series are used to infect the culture cells with the viruses. A TCID₅₀ value (50% tissue culture infectious dose) is determined to quantify the viruses. Specifically, the antiviral test is preferably a test performed according to the method described in, for example, ISO 21702 (2019). Note that, the ISO 21702 (2019) is a test for viruses obtained by modification of the aforementioned antimicrobial tests: ISO 22196 and JIS Z 2801.

When the test is performed according to the method of ISO 21702 (2019), the case where the antiviral activity value evaluated in the present test is 0.2 or more is preferably judged as having an antiviral activity. The antiviral activity value is preferably 0.5 or more, more preferably 1.0 or more, still more preferably 1.5 or more, particularly preferably 2.0 or more. Here, the antiviral activity value obtained according to the method of ISO 21702 (2019) is represented by the following numerical formula. Specifically, the same virus culture is each inoculated into a test piece X and a test piece Y, and the viral infectivity titer (PFU/cm²) obtained after 24 hours is measured, to calculate the antiviral activity value based on the following numerical formula. The case where the antiviral activity value is 2 or more may be defined as an antivirus material. However, in the present embodiment, the case where the antiviral activity value is 0.2 or more is judged as “having an antiviral activity” in terms of preventing proliferation of viruses.

Antiviral activity value=Ut−At
Ut: An average value of common logarithm values of viral infectivity titers on test piece Y obtained after 24 hours
At: An average value of common logarithm values of viral infectivity titers on test piece X obtained after 24 hours

The anti-pathogen structure preferably has a water resistance. Specifically, the anti-pathogen structure more preferably has an anti-pathogen activity even when immersed in water (specifically, for example, purified water and ion exchanged water) of 25 degrees Celsius for 24 hours. The reason for this is because it is assumed that the anti-pathogen structure is used under an environment where water adheres when the anti-pathogen structure is used.

The microorganisms refer to small prokaryotes and eukaryotes. Examples of the microorganisms include: gram-negative bacteria and gram-positive bacteria, Staphylococcus aureus, Escherichia coli, Yersinia pestis, Vibrio cholerae, Mycobacterium tuberculosis, Pseudomonas aeruginosa, Spirochaeta that causes syphilis or Lyme disease, Rickettsia that causes epidemic louse-borne typhus or scrub typhus (tsutsugamushi disease), Chlamydia, Mycoplasma, and cyanobacteria, which are classified into the bacteria of the prokaryote; methanogens and hyperthermophiles, which are classified into the archaebacterial of the prokaryote; and molds, fungi, yeast, Candida, Trichophyton, and Plasmodium that causes malaria, which are classified into the eukaryote.

The microorganisms in the present application are not limited to the microorganism that have been currently identified, and also include microorganisms that will be identified in the future. Examples of the microorganisms that will be identified in the future include pharmaceutical agent-resistant bacteria such as MRSA (Methicillin-resistant Staphylococcus aureus) and microorganisms that will be newly identified or named.

The viruses are extremely small infectious structures that replicate themselves by using a cell of another organism. Examples of the viruses include: DNA viruses including, for example, herpesvirus, poxvirus, and hepadnavirus; and RNA viruses including, for example, flavivirus, togavirus, coronavirus, Hepatitis D virus, orthomyxovirus, paramyxovirus, rhabdovirus, bunyavirus, filovirus, and retrovirus.

Examples of the orthomyxovirus include influenza virus A, influenza virus B, influenza virus C, isavirus, thogoto virus, and quaranjavirus.

Examples of the coronavirus include alphacoronavirus, betacoronavirus, gammacoronavirus, and deltacoronavirus.

Examples of the paramyxovirus include paramyxovirus, rubulavirus, morbillivirus, and pneumovirus.

The viruses in the present application are not limited to the viruses that have been currently identified, and also include viruses that will be identified in the future. Examples of the viruses that will be identified in the future include mutated new viruses and viruses that will be newly identified or named.

A shape of the anti-pathogen structure may be appropriately selected depending on the intended purpose. Examples of the shape include shapes such as a layer shape (film shape) and a particulate shape. When the anti-pathogen structure having a layer shape (film shape) is used, the layer shape (film shape) may be a flat shape or a curved shape. A composite formed by gathering a plurality of anti-pathogen structures or a coated matter formed on the surface of another member may be used.

At the time of use, the shape of the anti-pathogen structure is preferably such a shape that the surface structure is easily affected by, for example, abrasion. The reason for this is as follows. Specifically, even when having such a shape, the anti-pathogen structure of the present disclosure has a high durability, and an effect of preventing the antimicrobial activity or the antiviral activity from being decreased can be further significantly achieved. At the time of such a use, such a shape that the surface structure is easily affected by, for example, abrasion may be, for example, a layer shape (film shape). Meanwhile, in the case of the particulate shape, the surface structure has such a shape that the surface structure is hardly affected by, for example, abrasion at the time of use. Therefore, the shape of the anti-pathogen structure may not be a particulate shape.

<Resin Structure>

The resin structure is a structure formed using a resin as a material. The resin structure refers to: a structure including, as a material, a synthetic resin produced by artificially allowing a polymerizable compound to polymerize; or a structure including, as a material, a naturally derived resin produced by artificially processing or treating a natural resin derived from a plant or an animal. The resin structure does not include a structure including only an unprocessed or untreated material such as a natural resin. The resin structure according to the present embodiment per se exhibits the anti-pathogen activity, as described above.

On the surface of the resin structure, a surface structure including a plurality of openings is formed. When microorganisms or viruses contact the surface structure, the surface adsorption force originated from the openings destroys outer shell parts of the microorganisms or the viruses, and the anti-pathogen activity is expressed. In the present action, even an anti-pathogen structure, which does not substantially contain an antimicrobial agent or an antiviral agent that is a pharmaceutical agent, can exhibit the anti-pathogen activity. This makes it possible to prevent an influence on a human body that may be caused by the antimicrobial agent or the antiviral agent (e.g., allergic reaction). Unlike the antimicrobial agent or the antiviral agent, it is not consumed over time, and thus the persistence of the effect (anti-pathogen activity) is improved. Moreover, occurrence of microorganisms or viruses having a tolerance to the antimicrobial agent or the antiviral agent can be prevented.

The surface structure includes a plurality of openings and a skeleton that shapes the plurality of openings. The openings are parts other than the skeleton in the surface structure, and represent at least space open to the outside. The skeleton is a part other than the plurality of openings in the surface structure, and represents a structure part formed of a resin. The skeleton is a continuous structure on the surface of the resin structure, and the continuous structure shapes the plurality of openings. Therefore, a conventional structure, which has a pillar with fine projection structures (nanopillar) but has a surface structure that is not continuous, has a low durability. Meanwhile, the surface structure of the present embodiment is not easily affected by deterioration of the fine structure caused by abrasion. Therefore, the durability of the anti-pathogen structure is improved. That is, the surface structure according to the present embodiment easily maintains a shape of the plurality of openings contributing to achievement of the anti-pathogen activity, to prevent the anti-pathogen activity from being decreased.

A shape of the opening is not particularly limited. Examples of the shape include various shapes such as a substantially circular shape, a substantially elliptic shape, and a substantially polygonal shape. A pore diameter of the openings is not particularly limited. The pore diameter of the openings refers to a length of the longest straight line drawn when the opening is observed (in other words, when the opening is viewed in plan). Specifically, the pore diameter of the openings can be determined using a photograph taken by, for example, a scanning electron microscope (SEM).

In order to achieve an antimicrobial activity, the pore diameter of the openings is preferably 10 micrometers or less, more preferably 5 micrometers or less, still more preferably 1 micrometer or less, particularly preferably 0.5 micrometers or less. When the pore diameter of the openings is 10 micrometers or less, the surface adsorption force originated from the openings destroys outer shell parts (e.g., cell membrane and cell wall) of microorganisms, and the antimicrobial activity is appropriately expressed. Preferably, the pore diameter of the openings is appropriately changed depending on a kind or size of a microorganism in which the antimicrobial activity is to be exhibited. Generally, the pore diameter of the openings is preferably smaller than the maximum diameter of a microorganism. For example, the pore diameter of the openings is preferably 10 micrometers or less in the case of a fungus, the pore diameter of the openings is preferably 1 micrometer or less in the case of Staphylococcus aureus, and the pore diameter of the openings is preferably 4 micrometers or less in the case of Escherichia coli. The surface adsorption force is inversely proportional to the pore diameter of the openings. Therefore, the smaller the pore diameter is, the larger the surface adsorption force is, and therefore a higher antimicrobial activity can be expected. The pore diameter of the openings can be appropriately adjusted by, for example, polymerization conditions (e.g., irradiation intensity and irradiation time of active energy rays to be emitted) under which the polymerizable compound is allowed to polymerize. In order to distinguish the pore diameter of the openings for the purpose of achieving the following antiviral activity, the pore diameter of the openings for the purpose of achieving the antimicrobial activity may be larger than 0.1 micrometers.

In order to achieve an antiviral activity, the pore diameter of the openings is preferably 0.1 micrometers or less, more preferably 0.05 micrometers or less. When the pore diameter of the openings is 0.1 micrometers or less, the surface adsorption force originated from the openings destroys outer shell parts (e.g., envelope) of viruses, and the antiviral activity is appropriately expressed. Preferably, the pore diameter of the openings is appropriately changed depending on a kind or size of a virus in which the antiviral activity is to be exhibited. Generally, the pore diameter of the openings is preferably smaller than the maximum diameter of a virus. The surface adsorption force is inversely proportional to the pore diameter of the openings. Therefore, the smaller the pore diameter is, the larger the surface adsorption force is, and therefore a higher antiviral activity can be expected. The pore diameter of the openings can be appropriately adjusted by, for example, polymerization conditions (e.g., irradiation intensity and irradiation time of active energy rays to be emitted) under which the polymerizable compound is allowed to polymerize. Specifically, the pore diameter of the openings can be decreased by, for example, increasing an amount of the polymerizable compound or enhancing irradiation intensity of active energy rays to be emitted. The lower limit of the pore diameter of the openings in the case where the antiviral activity is to be exhibited is not particularly limited, but is preferably, for example, 0.001 micrometers or more.

A shape of the skeleton is not particularly so long as it can shape the opening. Examples of the shape include various shapes such as a shape obtained by coupling a plurality of particles to each other and a substantially plane shape. FIG. 2 is a view obtained by observing, with SEM, the surface of the resin structure (anti-pathogen structure) that includes: a skeleton having a shape obtained by coupling a plurality of particles to each other; and an opening shaped by the skeleton. FIG. 3 is a view obtained by observing, with SEM, the surface of the resin structure (anti-pathogen structure) that includes: a skeleton having a substantially plane shape; and an opening shaped by the skeleton. As the shape of the skeleton, the substantially plane shape is more preferable than the shape obtained by coupling a plurality of particles to each other. The reason for this is as follows. Specifically, in the case of the substantially plane shape, the surface on which the surface structure of the resin structure is formed has a high hardness, an influence caused by deteriorating fine structure due to abrasion can be decreased, and the durability of the anti-pathogen structure is improved. As a result, the anti-pathogen activity can be prevented from being decreased. Regarding the hardness of the surface on which the surface structure of the resin structure is formed, the pencil hardness in the evaluation according to the method described in, for example, ISO 15184 is preferably used. At this time, when the skeleton has a shape obtained by coupling a plurality of particles to each other, the pencil hardness is from 6B through 2B. Meanwhile, when the shape of the skeleton is a substantially plane shape, the pencil hardness can be B or harder, and further can be F or harder. This evaluation can be performed by application of load (750 g) using, for example, a pencil hardness tester (available from Toyo Seiki Seisaku-sho, Ltd.). Moreover, in order to improve the durability of the anti-pathogen structure and to prevent the anti-pathogen activity from being decreased, the pencil hardness is preferably a high hardness.

The resin structure preferably has a porous structure having a co-continuous structure in which a plurality of pores are continuously coupled to each other. More preferably, the plurality of openings are each independently coupled to some of the pores constituting the co-continuous structure.

As described above, the resins structure includes a plurality of pores therein, and is preferably such a structure that these pores are coupled to each other (in other words, the plurality of pores are continuously coupled to each other). Such a structure is also called a co-continuous structure or a monolith structure. Because the resin structure includes numerous pores and one pore is coupled to another pore around the pore, it has a communication property, and the continuous pores spread three-dimensionally. When the plurality of openings are each independently coupled to some of the pores constituting the co-continuous structure, continuous capillarity from the openings in the surface to the inner co-continuous structure is expressed, further improving the anti-pathogen activity. Moreover, the dead bodies of microorganisms or viruses are discharged from the opening in the surface to the inner co-continuous structure, and are prevented from remaining on the surface. Therefore, the anti-pathogen activity over time can be prevented from being decreased. Moreover, even when the surface of the resin structure is shaved, the inner pores are exposed as new openings, to exhibit the anti-pathogen activity. Therefore, the expected effects of the anti-pathogen structure continue for a long period of time compared to conventional structures having a pillar with fine projection structures (nanopillar).

Examples of a method for confirming that pores are coupled to each other include a method where an image of a cross section of the resin structure is observed with, for example, a scanning electron microscope (SEM) to confirm that the pores coupled to each other are continuous. One example of the physical characteristic obtained when pores are coupled to each other is, for example, air permeability. The air permeability of the resin structure is measured according to, for example, JIS P8117. The air permeability of the resin structure is preferably 1,000 seconds/100 mL or less, more preferably 500 seconds/100 mL or less, still more preferably 300 seconds/100 mL or less. At this time, the air permeability is measured using, for example, a gurley-type densometer (available from Toyo Seiki Seisaku-sho, Ltd.). As one example, when the air permeability is 1,000 seconds/100 mL or less, it may be judged that the pores are coupled to each other.

The porosity of the resin structure is preferably 10% or more, more preferably 15% or more, still more preferably 30% or more, particularly preferably 50% or more. In addition, the porosity of the resin structure is preferably 90% or less. When the porosity is 30% or more, continuous capillarity from the openings in the surface to the inner co-continuous structure is further expressed, further improving the anti-pathogen activity. When the porosity is 90% or less, strength of the resin structure is improved. A method for measuring the porosity of the resin structure is not particularly limited. One example of the method is as follows, for example. Specifically, the resin structure is loaded with an unsaturated fatty acid (commercially available butter) and is subjected to the osmium staining. Then, the inner cross-sectional structure is cut through FIB, and the porosity of the anti-pathogen structure is measured with SEM.

A shape of a cross section of the pore in the resin structure is not particularly limited. Examples of the shape include various shapes such as a substantially circular shape, a substantially elliptic shape, and a substantially polygonal shape. A pore diameter of the pore is not particularly limited as well. Here, the pore diameter of the pore refers to a length of the longest straight line drawn in the cross-sectional shape. Specifically, the pore diameter of the pore can be determined using a photograph of the cross section taken by, for example, a scanning electron microscope (SEM).

In order to achieve the antimicrobial activity, the pore diameter of the pore of the resin structure is preferably 10 micrometers or less, more preferably 5 micrometers or less, still more preferably 1 micrometer or less, particularly preferably 0.5 micrometers or less. When the pore diameter of the pore is 10 micrometers or less, outer shell parts (e.g., cell membrane and cell wall) of microorganisms are more easily destroyed in the openings coupled to each other by capillarity originated from the pores, and the antimicrobial activity is appropriately expressed. Here, the capillarity is inversely proportional to the pore diameter of the pore. Therefore, the smaller the pore diameter is, the larger the capillarity is, and therefore a higher antimicrobial activity can be expected. The pore diameter of the openings can be appropriately adjusted by, for example, polymerization conditions (e.g., irradiation intensity and irradiation time of active energy rays to be emitted) under which the polymerizable compound is allowed to polymerize.

—Material Constituting Resin Structure—

The resin that is a material constituting the resin structure will be described.

One example of a usable resin is not particularly limited. Examples thereof include: resins (e.g., acrylate resins, methacrylate resins, urethane acrylate resins, vinyl ester resins, unsaturated polyester resins, epoxy resins, oxetane resins, and vinyl ether resins) that can be formed by irradiation of active energy rays such as ionizing radiation, ultraviolet rays, and infrared rays (heat); and resins that utilize ene-thiol reaction. Among them, acrylate resins, methacrylate resins, urethane acrylate resins, and vinyl ester resins, which can be formed using the highly reactive radical polymerization, are preferable, and acrylate resins and (meth)acrylic resins such as methacrylate resins are more preferable.

Another example of the usable resin is not particularly limited. Examples thereof include biodegradable resins and thermoplastic resins. Preferable examples of the biodegradable resin include aliphatic polyester resins. Examples of the aliphatic polyester resin include polylactic acid/glycolic acid copolymer (PLGA), polylactic acid (PLA), poly-ε-caprolactone, succinate polymer, and polyhydroxyalkanoate. Examples of the thermoplastic resin include polyvinylidene fluoride (PVDF), polyethylene, polypropylene, polystyrene, acrylic resins, polyvinyl chloride, polyvinyl acetate, ABS resins, polyamide, polyester, polycarbonate, Teflon (registered trademark), polyimide, and polysulphone.

A method for producing the resin structure including a plurality of openings in the surface thereof using the aforementioned materials is not particularly limited. Examples of the method include methods such as induced phase separation using heat or light, track etching using laser, foaming using gas, drawing of a film, and use of a good solvent and a poor solvent for a resin. Among them, the method utilizing induced phase separation using heat or light and the method utilizing a good solvent and a poor solvent for a resin are preferable. Therefore, these methods will be described hereinafter.

—Liquid Composition that Forms Resin Structure Through Curing—

A liquid composition that is cured through polymerization to form a resin constituting the resin structure (also referred to as “curing-type composition”) preferably includes a polymerizable compound, a solvent, a polymerization initiator, and an organic polymeric compound. In the resin structure formed of the liquid composition, a surface structure including a plurality of openings is preferably formed at the time of curing. More preferably, in the resin structure formed of the liquid composition, the surface structure including the plurality of openings is formed at the time of curing, and a co-continuous structure obtained by coupling the openings to each other is formed at the same time. The present method is more advantageous because the anti-pathogen structure can be produced in a short process time compared to a structure including a pillar with fine projection structures (nanopillar), which requires a long process time for production (e.g., a transfer method such as nanoimprint, and patterning). Furthermore, the present method is more advantageous because of the following reason. That is, because the liquid composition can be discharged on an object (base material) to which the anti-pathogen activity is to be achieved by, for example, an inkjet method and a spray method, the anti-pathogen structure can be produced on the object (base material) in a non-contact manner, compared to the transfer method such as nanoimprint. More specifically, production of the anti-pathogen structure in a non-contact manner is advantageous in the following cases: a case where the transfer method cannot be applied because an object (base material) is structurally weak; a case where an object (base material) has a complex three-dimensional shape such as a curve structure; and a case where an object (base material) needs to be treated in a non-contact manner in terms of, for example, sanitation. Whether the liquid composition forms a resin structure having predetermined shape and characteristics is judged based on a structure formed according to the following method. First, a liquid composition (20 μl/cm²) is applied to a glass plate so as to form a solid image. Immediately after that, under N₂ atmosphere, the applied region of the liquid composition is irradiated with ultraviolet rays (UV) (light source: UV-LED (available from Phoseon, product name: FJ800), wavelength: 365 nm, irradiation intensity: 30 mW/cm², irradiation time: 20 s) to cure the applied region of the liquid composition. As a result, a structure is obntained.

——Polymerizable Compound——

The polymerizable compound forms a resin through polymerization, and forms a porous resin having the opening and the pore when polymerizing in a liquid composition. The polymerizable compound preferably forms a resin by irradiation of active energy rays. The resin formed of the polymerizable compound preferably has a crosslinked structure in a molecule thereof by using a bifunctional or higher polymerizable compound. This makes it possible to increase a glass transition temperature or a melting point of the resin, which results in improvement of the strength. In addition, the crosslinked structure also improves the water resistance.

The active energy rays are not particularly limited so long as the active energy rays can give necessary energy to allow polymerization reaction of the polymerizable compound in the liquid composition to proceed. Examples of the active energy rays include ultraviolet rays, electron beams, α-rays, β-rays, γ-rays, and X-rays. Among them, ultraviolet rays are preferable. When a light source with a particularly high energy is used, polymerization reaction can proceed without using a polymerization initiator.

The polymerizable compound preferably includes at least one radical polymerizable functional group. Examples thereof include monofunctional radical polymerizable compounds, bifunctional radical polymerizable compounds, trifunctional or higher radical polymerizable compounds, functional monomers, and radical polymerizable oligomers. Among them, bifunctional or higher radical polymerizable compounds are preferable.

Preferable examples of the polymerizable compound include polymerizable compounds having a (meth)acryloyl group or a vinyl group.

Examples of the monofunctional radical polymerizable compound include 2-(2-ethoxyethoxy)ethylacrylate, methoxy polyethylene glycol monoacrylate, methoxy polyethylene glycol monomethacrylate, phenoxy polyethylene glycolacrylate, 2-acryloyloxyethylsuccinate, 2-ethylhexylacrylate, 2-hydroxyethylacrylate, 2-hydroxypropylacrylate, tetrahydrofurfuryl acrylate, 2-ethylhexylcarbitolacrylate, 3-methoxybutylacrylate, benzyl acrylate, cyclohexyl acrylate, isoamyl acrylate, isobutyl acrylate, methoxytriethylene glycol acrylate, phenoxytetraethylene glycol acrylate, cetyl acrylate, isostearyl acrylate, stearyl acrylate, and a styrene monomer. These may be used alone or in combination.

Examples of the bifunctional radical polymerizable compound include 1,3-butanedioldiacrylate, 1,4-butanedioldiacrylate, 1,4-butanedioldimethacrylate, 1,6-hexanedioldiacrylate, 1,6-hexanedioldimethacrylate, diethylene glycol diacrylate, polyethylene glycol diacrylate, neopentyl glycol diacrylate, EO-modified bisphenol A diacrylate, EO-modified bisphenol F diacrylate, neopentyl glycol diacrylate, and tricyclodecane dimethanol diacrylate. These may be used alone or in combination.

Examples of the trifunctional or higher radical polymerizable compound include trimethylolpropane triacrylate (TMPTA), trimethylolpropane trimethacrylate, EO-modified trimethylolpropane triacrylate, PO-modified trimethylolpropane triacrylate, caprolactone-modified trimethylolpropane triacrylate, HPA-modified trimethylolpropane trimethacrylate, pentaerythritol triacrylate, pentaerythritol tetraacrylate (PETTA), glycerol triacrylate, ECH-modified glycerol triacrylate, EO-modified glycerol triacrylate, PO-modified glycerol triacrylate, tris(acryloxyethyl) isocyanurate, dipentaerythritol hexaacrylate (DPHA), caprolactone-modified dipentaerythritol hexaacrylate, dipentaerythritol hydroxypentaacrylate, alkyl-modified dipentaerythritol pentaacrylate, alkyl-modified dipentaerythritol tetraacrylate, alkyl-modified dipentaerythritol triacrylate, dimethylol propane tetraacrylate (DTMPTA), pentaerythritol ethoxy tetraacrylate, EO-modified phosphoric acid triacrylate, and 2,2,5,5-tetrahydroxymethylcyclopentanonetetraacrylate. These may be used alone or in combination.

An amount of the polymerizable compound in the liquid composition is preferably 5.0% by mass or more but 70.0% by mass or less, more preferably 10.0% by mass or more but 50.0% by mass or less, still more preferably 20.0% by mass or more but 50.0% by mass or less, relative to the total amount of the liquid composition. The amount of the polymerizable compound satisfying 70.0% by mass or less is preferable because a size of an opening or a pore of the resin structure to be obtained can fall within an appropriate range. The amount of the polymerizable compound satisfying 5.0% by mass or more is preferable because strength of the resin structure is improved.

——Solvent——

The solvent (also referred to as “porogen” hereinafter) is liquid compatible with a polymerizable compound.

The solvent (also referred to as “porogen” hereinafter) is liquid that is compatible with the polymerizable compound. The solvent is liquid that becomes incompatible with the polymer (resin) (phase separation occurs) in the process of allowing the polymerizable compound to polymerize in a liquid composition. That is, the meaning of the “solvent” in the present disclosure is distinguished from the meaning of the generally used term “solvent”. Inclusion of the solvent in the liquid composition makes it possible to form a porous resin having the aforementioned openings and pores when the polymerizable compound polymerizes in the liquid composition. In addition, the solvent can preferably dissolve a compound that generates a radical or an acid by application of light or heat (i.e., a polymerization initiator that will be described hereinafter). The solvent may be used alone or two or more kinds of solvents may be used in combination. Note that, the solvent has no polymerization ability.

The boiling point of the porogen used alone or the boiling points of two kinds of porogens used in combination are preferably 50 degrees Celsius or more but 250 degrees Celsius or less, more preferably 70 degrees Celsius or more but 200 degrees Celsius or less at normal pressure. When the boiling point is 50 degrees Celsius or more, vaporization of the porogen at nearly room temperature can be prevented, the liquid composition is easily handled, and an amount of the porogen contained in the liquid composition can be easily controlled. When the boiling point is 250 degrees Celsius or less, time required in a step of drying the porogen after polymerization is shortened, to improve productivity of the resin structure.

Examples of the porogen include: ethylene glycols such as diethylene glycol monomethyl ether, ethylene glycol monobutyl ether, ethylene glycol monoisopropyl ether, triethylene glycol monobutyl ether, and dipropylene glycol monomethyl ether; esters such as γ butyrolactone and propylene carbonate; and amides such as NN dimethylacetamide.

Examples of the porogen include liquids having a relatively large molecular weight (e.g., methyl tetradecanoate, methyl decanoate, methyl myristate, and tetradecane). Moreover, liquids such as acetone, 2-ethylhexanol, and 1-bromonaphthalene can also be used.
Note that, the aforementioned exemplified liquids do not always correspond to the porogen. As described above, the porogen is liquid, which is compatible with the polymerizable compound and becomes incompatible with the polymer (resin) (phase separation occurs) in the process of allowing the polymerizable compound to polymerize in a liquid composition. In other words, whether one liquid corresponds to the porogen can be determined by a relationship between a polymerizable compound and a polymer (a resin formed by allowing the polymerizable compound to polymerize).
Note that, the liquid composition may include at least one kind of porogen satisfying the aforementioned certain relationship between the polymerizable compound and the polymer as described above. Therefore, liquid that does not satisfy the aforementioned certain relationship between the polymerizable compound and the polymer (i.e., liquid that is not the porogen) may be additionally contained. An amount of the liquid that does not satisfy the aforementioned certain relationship between the polymerizable compound and the polymer (i.e., liquid that is not the porogen) is preferably 10.0% by mass or less, more preferably 5.0% by mass or less, still more preferably 1.0% by mass or less, relative to the total amount of the liquid composition. Particularly preferably, the liquid that does not satisfy the aforementioned certain relationship between the polymerizable compound and the polymer (i.e., liquid that is not the porogen) is not contained.

An amount of the porogen in the liquid composition is preferably 30.0% by mass or more but 95.0% by mass or less, more preferably 50.0% by mass or more but 90.0% by mass or less, still more preferably 50.0% by mass or more but 80.0% by mass or less, relative to the total amount of the liquid composition. The amount of the porogen satisfying 30.0% by mass or more is preferable because a size of the opening or the pore of the resin structure obtained can fall within an appropriate range. The amount of the porogen satisfying 95.0% by mass or less is preferable because strength of the resin structure can be improved.

A mass ratio (polymerizable compound:porogen) between the amount of the polymerizable compound and the amount of the porogen in the liquid composition is preferably from 1.0:0.4 through 1.0:19.0, more preferably from 1.0:1.0 through 1.0:9.0, still more preferably from 1.0:1.0 through 1.0:4.0.

——Polymerization Initiator——

The polymerization initiator is a material that can generate active species such as a radical or a cation by application of energy such as light or heat, to initiate polymerization of the polymerizable compound. As the polymerization initiator, radical polymerization initiators, cationic polymerization initiators, and base generators known in the art can be used alone or in combination. Among them, a photo-radical polymerization initiator is preferably used.

As the photo-radical polymerization initiator, a photo-radical generator can be used. Examples thereof include photo-radical polymerization initiators such as Michler's ketone and benzophenone, which are known as product names: IRGACURE and DAROCUR. As more specific compounds, it is suitable to use, for example, benzophenone, acetophenone derivatives (e.g., α-hydroxyacetophenone and xaminoacetophenone), 4-aroyl-1,3-dioxolane, benzyl ketal, 2,2-diethoxyacetophenone, p-dimethylaminoacetophenone, p-dimethylamino propiophenone, benzophenone, 2-chlorobenzophenone, pp′-dichlorobenzophenone, pp′-bisdiethylamino benzophenone, Michler's ketone, benzyl, benzoin, benzyl dimethyl ketal, tetramethylthiuram monosulfide, thioxanthone, 2-chlorothioxanthone, 2-methylthioxanthone, azobisisobutyronitrile, benzoin peroxide, di-tert-butylperoxide, 1-hydroxycyclohexyl phenyl ketone, 2-hydroxy-2-methyl-1-phenyl-1-one, 1-(4-isopropylphenyl)-2-hydroxy-2-methylpropan-1-one, methyl benzoylformate, benzoin isopropyl ether, benzoin methyl ether, benzoin ethyl ether, benzoin ether, benzoin isobutyl ether, benzoin n-butyl ether, benzoin n-propyl, 1-hydroxycyclohexyl phenyl ketone, 2-benzyl 2-dimethylamino 1-(4-morpholinophenyl)-butanone-1, 1-hydroxycyclohexyl-phenyl-ketone, 2,2-dimethoxy-1,2-diphenylethan-1-one, bis(η5-2,4-cyclopentadien-1-yl)-bis(2,6-difluoro-3-(1H-pyrrol-1-yl)-phenyl)titanium, bis(2,4,6-trimethylbenzoyl)-phenylphosphine oxide, 2-methyl-1[4-(methylthio)phenyl]-2-morpholinopropan-1-one, 2-hydroxy-2-methyl-1-phenyl-propan-1-one (DAROCUR 1173), bis(2,6-dimethoxybenzoyl)-2,4,4-trimethyl-pentylphosphine oxide, 1-[4-(2-hydroxyethoxy)-phenyl]-2-hydroxy-2-methyl-1-propan-1-one monoacylphosphine oxide, bisacylphosphine oxide or titanocene, fluorescein, anthraquinone, thioxanthone or xanthone, lophine dimer, trihalomethyl compounds or dihalomethyl compounds, active ester compounds, and organic boron compounds.

A photo-crosslinkable radical generator such as a bisazido compound may be contained at the same time. Alternatively, a thermal polymerization initiator such as azobisisobutyronitrile (AIBN), which is a general radical generator, may be used when polymerization is performed by application of heat.

When the total mass of the polymerizable compound is 100.0% by mass, an amount of the polymerization initiator is preferably 0.05% by mass or more but 10.0% by mass or less, more preferably 0.5% by mass or more but 5.0% by mass or less, in order to obtain a sufficient curing rate.

——Organic Polymeric Compound——

The organic polymeric compound is an organic compound, which is polymeric and is to be added to the liquid composition. Examples of the organic polymeric compound include resins (also referred to as “addition resin” hereinafter) and organic polymeric compounds originated from natural products. Note that, the addition resin is different from a resin formed by polymerization of the polymerizable compound (may be called “polymerizable resin”). The organic polymeric compound is preferably added to the liquid composition, but may not be added thereto. The organic polymeric compound preferably does not include a polymerizable functional group.

Addition of the organic polymeric compound to a liquid composition can improve hardness of the resin structure formed by curing the liquid composition. The reason why the organic polymeric compound can improve the hardness of the resin structure will be described below.

The following can be assumed. Generally, in the process of curing a liquid composition containing no organic polymeric compound, a resin formed by polymerization of the polymerizable compound becomes insoluble with the liquid composition as polymerization proceeds, to form particulate nuclei. The nuclei are gathered and bound through the intermolecular force. As a result, it is possible to form a resin structure that includes a skeleton having a shape obtained by coupling a plurality of particles to each other. Meanwhile, in the process of curing a liquid composition containing an organic polymeric compound, a bonding strength that enables reversible binding and dissociating occurs between the organic polymeric compound and the polymerizable compound, to allow polymerization of the polymerizable compound to proceed along a long chain of the organic polymeric compound (in other words, a bonding strength that enables reversible binding and dissociating occurs even between a polymer of the polymerizable compound and the organic polymeric compound). As a result, as polymerization proceeds, the resin formed by polymerization of the polymerizable compound becomes insoluble with the liquid composition. Then, formation of particulate nuclei is prevented, to form a resin structure that includes a skeleton having a substantially plane shape. As described above, the hardness of a resin structure that includes a skeleton having a substantially plane shape is higher than the hardness of a resin structure that includes a skeleton having a shape obtained by coupling a plurality of particles to each other. Therefore, it can be said that the organic polymeric compound can improve the hardness of the resin structure.

Note that, the bonding strength that enables reversible binding and dissociating is preferably a hydrogen bond (2 kJ/mol to 40 kJ/mol). That is, the organic polymeric compound preferably includes a functional group that can bind the polymerizable compound and a polymer of the polymerizable compound via a hydrogen bond. The organic polymeric compound is preferably dissolved in a solvent. The reason for this is because when the organic polymeric compound can be dissolved in the solvent, polymerization of the polymerizable compound appropriately proceeds along a long chain of the organic polymeric compound. Here, the phrase “an organic polymeric compound is dissolved in a solvent” means that when 20 g of an organic polymeric compound is added to 100 g of a solvent (25 degrees Celsius), followed by mixing and stirring, 90% by mass or more of the organic polymeric compound is dissolved.

The addition resin is not particularly limited so long as a bonding strength that enables reversible binding and dissociating occurs between the polymerizable compound and a polymer of the polymerizable compound. Examples of the addition resin include a resin including a hydroxyl group in a molecule thereof because it preferably includes a functional group that can form a hydrogen bond. Specific examples thereof include polyacryl polyol, polyester polyol, polybutadiene polyol, polyvinyl butyral, polyvinyl acetal, ethyl cellulose, and nitrocellulose. Among them, for example, polyvinyl butyral is preferable. These may be used alone or in combination. Moreover, an appropriately synthesized product or a commercially available product may be used.

The organic polymeric compound originated from a natural product is not particularly limited so long as the bonding strength that enables reversible binding and dissociating occurs between the polymerizable compound and a polymer of the polymerizable compound. The organic polymeric compound originated from the natural product preferably includes a functional group that can form a hydrogen bond. Specifically, preferable examples thereof include lignin derivatives originated from natural lignin.

Examples of the natural lignin include lignin contained in natural wood and lignin contained in herbaceous plants such as rice straw and wheat straw.

The lignin derivatives can be obtained by subjecting natural lignin to, for example, a predetermined treatment as described below.

As one example of the predetermined treatment, such a treatment method that lignin is removed from natural wood to obtain pulp is a representative treatment method. One example thereof is a pulp treatment using a Kraft process. This is a method using, as a cooking liquor, a sodium hydroxide aqueous solution and a sodium sulfide aqueous solution. When a molecular-weight-reducing treatment is performed in order to separate lignin from natural wood, a lignin derivative can be obtained. The lignin derivative obtained in this treatment is referred to as “kraft lignin”.

One example of another treatment is as follows. Specifically, a material such as wood is saccharified using sulfuric acid to obtain a residue lignin. Then, the residue lignin is subjected to a hydrothermal treatment in an alkali aqueous solution for water solubilization, to obtain a lignin derivative. The lignin derivative obtained by the present treatment is referred to as a “hydrothermally treated sulfuric acid lignin”.
As one example of another treatment, an herbaceous plant material such as rice straw or wheat straw is treated in an alkali aqueous solution for water solubilization, to obtain a lignin derivative. The lignin derivative obtained in this treatment is referred to as “alkali lignin”.
In addition, an enzymatically saccharified lignin can also be used.
The lignin derivative in the present disclosure is not limited to those obtained after the aforementioned predetermined treatments. Such lignin to which an additional treatment (e.g., a hydroxymethylation treatment and a phosphorylation treatment) has been subjected after the aforementioned predetermined treatment may be used.

When the total mass of the liquid composition is 100.0% by mass, an amount of the organic polymeric compound is preferably 1.0% by mass or more but 15.0% by mass or less, more preferably 1.3% by mass or more but 10.0% by mass or less, in order to obtain a sufficient hardness of the resin structure.

——Physical Property of Liquid Composition——

The viscosity of the liquid composition at 25 degrees Celsius is preferably 1.0 mPa·s or more but 200.0 mPa·s or less, more preferably 1.0 mPa·s or more but 150.0 mPa·s or less, still more preferably 1.0 mPa·s or more but 100.0 mPa·s or less, yet more preferably 1.0 mPa·s or more but 30.0 mPa·s or less, particularly preferably 1.0 mPa·s or more but 25.0 mPa·s or less, in terms of workability at the time of application of the liquid composition. The viscosity of the liquid composition satisfying 1.0 mPa·s or more but 200.0 mPa·s or less makes it possible to obtain a good dischargeability when the liquid composition is applied to a discharging method, preferably an inkjet method. Here, the viscosity can be measured using, for example, a viscometer (device name: RE-550L, available from Toki Sangyo Co., Ltd).

—Liquid Composition that Forms Resin Structure Through Drying—

A liquid composition (also referred to as a “precipitation-type composition”), which is dried to precipitate or aggregate (hereinafter “precipitate or aggregate” will be collectively referred to as simply “precipitate”) a dissolved or dispersed resin to form the resin structure, preferably includes, for example, a resin (hereinafter, also referred to as “precipitation resin”), a good solvent for the precipitation resin, and a poor solvent for the precipitation resin. In the resin structure formed of the liquid composition, a surface structure including a plurality of openings is preferably formed at the time of drying. More preferably, in the resin structure formed of the liquid composition, the surface structure including the plurality of openings and a co-continuous structure obtained by coupling the openings to each other are simultaneously formed at the time of drying. The present method is more advantageous because the anti-pathogen structure can be produced in a short process time compared to a structure including a pillar with fine projection structures (nanopillar), which requires a long process time for production (e.g., a transfer method such as nanoimprint, and patterning). Furthermore, the present method is more advantageous because of the following reason. That is, because the liquid composition can be discharged on an object (base material) to which the anti-pathogen activity is to be achieved by, for example, an inkjet method and a spray method, the anti-pathogen structure can be formed on the object (base material) in a non-contact manner, compared to the transfer method such as nanoimprint. More specifically, production of the anti-pathogen structure in a noncontact manner is advantageous in the following cases: a case where the transfer method cannot be applied because an object (base material) is structurally weak; a case where an object (base material) has a complex three-dimensional shape such as a curve structure; and a case where an object (base material) needs to be treated in a noncontact manner in terms of, for example, sanitation.

First, the reason why a liquid composition containing, for example, a precipitation resin, a good solvent, and a poor solvent is dried to form the resin structure will be described below.

When a precipitation resin is dissolved or dispersed in liquid containing a good solvent and a poor solvent to form a liquid composition, the precipitation resin is mainly dissolved or dispersed in the good solvent, and the precipitation resin does not substantially exist in the poor solvent. That is, such a state that the precipitation resin is unevenly distributed can be achieved in the liquid composition. When the liquid composition in this state is dried to precipitate the precipitation resin, the precipitation resin remains in a part where the good solvent exists, and voids form in a part where the poor solvent exists. As a result, the resin structure of the precipitation resin produced becomes a porous structure including a plurality of openings in the surface thereof.

——Resin (Precipitation Resin)——

The liquid composition is dried to precipitate the precipitation resin, and a porous resin including the opening and the pore is formed. As described above, the precipitation resin is dissolved or dispersed in the good solvent, and is not substantially dissolved or dispersed in the poor solvent.

A resin that can be used as the precipitation resin is not particularly limited so long as the resin is dissolved or dispersed in a good solvent and is not substantially dissolved or dispersed in a poor solvent. Examples of the resin include the biodegradable resins and the thermoplastic resins described above.

When the total mass of the liquid composition is 100.0% by mass, an amount of the precipitation resin is preferably 0.1% by mass or more but 20.0% by mass or less, more preferably 5.0% by mass or more but 15.0% by mass or less.

——Good Solvent——

The good solvent is liquid that can dissolve or disperse the precipitation resin. In the present disclosure, the good solvent preferably represents liquid that can dissolve or disperse the precipitation resin when the precipitation resin (0.1 g) is added to the liquid (100 g) of 25 degrees Celsius.

The good solvent is not particularly limited so long as it is liquid that can dissolve or disperse the precipitation resin. Examples of the good solvent include alcohol, ketone, ether, acetonitrile, and tetrahydrofuran.

Examples of the alcohol include alcohols having 1 or more but 4 or less carbon atoms. Examples of the alcohols having 1 or more but 4 or less carbon atoms include methanol, ethanol, propanol, and butanol.

Examples of the ketone include ketones having 3 or more but 6 or less carbon atoms. Examples of the ketones having 3 or more but 6 or less carbon atoms include acetone, methyl ethyl ketone, and cyclohexanone.

Examples of the ether include ethers having 2 or more but 6 or less carbon atoms. Examples of the ethers having 2 or more but 6 or less carbon atoms include dimethyl ether, methyl ethyl ether, and diethyl ether.

These may be used alone or in combination. When two or more kinds of good solvents are used, alcohol and ketone are preferably used in combination, and ethanol and acetone are more preferably used in combination.

An amount of the good solvent is not particularly limited so long as it is such an amount that the precipitation resin can be dissolved or dispersed. For example, when the total mass of the liquid composition is 100.0% by mass, the amount of the good solvent is preferably 30.0% by mass or more but 90.0% by mass or less, more preferably 40.0% by mass or more but 80.0% by mass or less.

——Poor Solvent——

The poor solvent is liquid that does not substantially dissolve or disperse the precipitation resin. In the present disclosure, the poor solvent is preferably liquid that can dissolve or disperse a mass of the precipitation resin when the precipitation resin is added to the liquid (100 g) of 25 degrees Celsius, where the mass is a half or less relative to a mass of the precipitation resin that a good solvent (100 g) of 25 degrees Celsius can dissolve or disperse when the precipitation resin is added to the good solvent.

The poor solvent is liquid that is compatible with the good solvent in a certain amount without being separated from the good solvent.

The poor solvent is not particularly limited, so long as it is liquid that does not substantially dissolve or disperse the precipitation resin and is compatible with the good solvent in a certain amount without being separated from the good solvent. Examples of the poor solvent include methanol, ethanol, and water. These may be used alone or in combination.

An amount of the poor solvent is not particularly limited so long as it is such an amount that the poor solvent can be dispersed in the good solvent. For example, when the total mass of the liquid composition is 100.0% by mass, the amount of the poor solvent is preferably 10.0% by mass or more but 60.0% by mass or less, more preferably 20.0% by mass or more but 50.0% by mass or less.

——Physical Property of Liquid Composition——

The viscosity of the liquid composition at 25 degrees Celsius is preferably 1.0 mPa·s or more but 200.0 mPa·s or less, more preferably 1.0 mPa·s or more but 150.0 mPa·s or less, still more preferably 1.0 mPa·s or more but 100.0 mPa·s or less, yet more preferably 1.0 mPa·s or more but 30.0 mPa·s or less, particularly preferably 1.0 mPa·s or more but 25.0 mPa·s or less, in terms of workability at the time of application of the liquid composition. The viscosity of the liquid composition satisfying 1.0 mPa·s or more but 200.0 mPa·s or less makes it possible to obtain a good dischargeability when the liquid composition is applied to a discharging method, preferably an inkjet method. Here, the viscosity can be measured using, for example, a viscometer (device name: RE-550L, available from Toki Sangyo Co., Ltd).

<Other Substances>

The anti-pathogen structure may include other substances if necessary, in addition to the resin structure. Examples of the other substances include antimicrobial agents and antiviral agents. Specific examples of the antimicrobial agent and the antiviral agent include: organic matters where substances themselves have an antimicrobial activity or an antiviral activity (e.g., pharmaceutical agents); substances that exhibit an antimicrobial activity or an antiviral activity over time (e.g., hypochlorous acid); inorganic matters having an antimicrobial activity or an antiviral activity (e.g., silver and copper); and inorganic matters having a function of decomposing an organic matter through photocatalytic reaction (e.g., titanium oxide and tungsten oxide). Note that, a starting material of the resin structure that remains after production (e.g., polymerizable compound) should not be included in the antimicrobial agent or the antiviral agent in the present disclosure.

Preferably, the anti-pathogen structure of the present embodiment does not substantially include an antimicrobial agent or an antiviral agent. More preferably, the anti-pathogen structure of the present embodiment is free of an antimicrobial agent and an antiviral agent. The reason for this is as follows. Specifically, when the anti-pathogen structure is free of an antimicrobial agent and an antiviral agent, an influence on a human body that may be caused by the antimicrobial agent or the antiviral agent (e.g., allergic reaction) can be prevented, and occurrence of microorganisms or viruses having a tolerance to the antimicrobial agent or the antiviral agent can be prevented. The phrase “does not substantially include an antimicrobial agent” represents any one of the following cases: a case where an amount of the antimicrobial agent is 1.0% by mass or less relative to the mass of the anti-pathogen structure; a case where an amount of the antimicrobial agent is 0.5% by mass or less relative to the mass of the anti-pathogen structure; a case where an amount of the antimicrobial agent is 0.1% by mass relative to the mass of the anti-pathogen structure; a case where an antimicrobial activity achieved by the antimicrobial agent cannot be observed; and a case where an amount of the antimicrobial agent is undetectable. The phrase “does not substantially include an antiviral agent” represents any one of the following cases: a case where an amount of the antiviral agent is 1.0% by mass or less relative to the mass of the anti-pathogen structure; a case where an amount of the antiviral agent is 0.5% by mass or less relative to the mass of the anti-pathogen structure; a case where an amount of the antiviral agent is 0.1% by mass relative to the mass of the anti-pathogen structure; a case where an antiviral activity achieved by the antiviral agent cannot be observed; and a case where an amount of the antiviral agent is undetectable. Note that, observation of the antimicrobial activity achieved by the antimicrobial agent, detection of the amount of the antimicrobial agent, observation of the antiviral activity achieved by the antiviral agent, and detection of the amount of the antiviral agent are each performed by a known means commonly used in the art.

<<Apparatus for Producing Anti-Pathogen Structure and Method for Producing Anti-Pathogen Structure>>

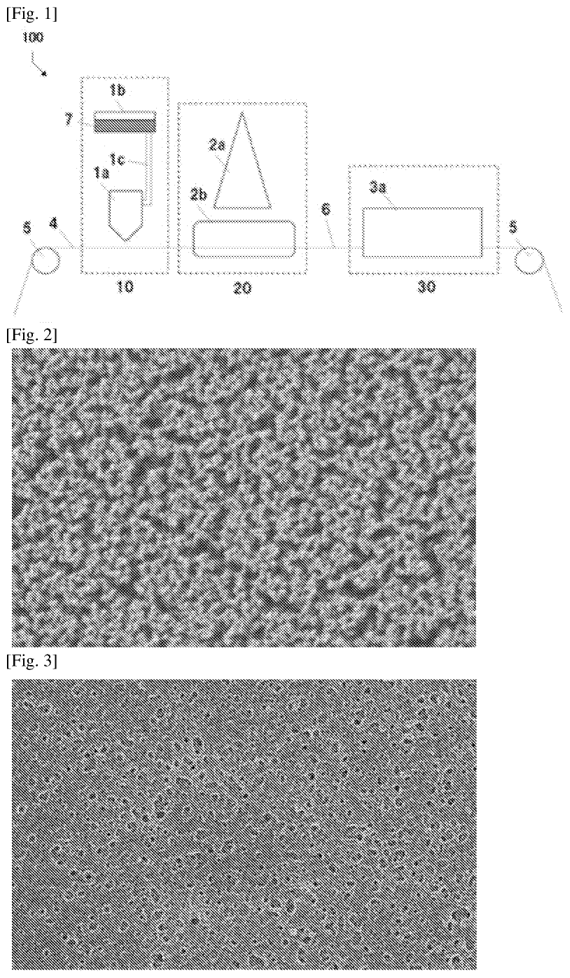

FIG. 1 is a schematic view presenting one example of an apparatus for producing an anti-pathogen structure in order to achieve a method for producing an anti-pathogen structure of an embodiment of the present disclosure. The production apparatus of FIG. 1 presents one example of an apparatus in the case where a liquid composition (curing-type composition), which forms a resin constituting a resin structure through polymerization and curing, is used. Even when a liquid composition (precipitation-type composition), which forms a resin structure by drying the liquid composition to precipitate a dissolved or dispersed resin, is used, the production apparatus of FIG. 1 can be applied by adding, deleting, and changing the configurations in the production apparatus of FIG. 1. Therefore, the production apparatus of FIG. 1 will be described below.

<Apparatus for Producing Anti-Pathogen Structure>

An apparatus for producing an anti-pathogen structure 100 is an apparatus for producing an anti-pathogen structure using the aforementioned liquid composition. The apparatus for producing an anti-pathogen structure 100 includes: an application step part 10; a polymerization step part 20; and a heating step part 30. The application step part 10 is configured to apply a liquid composition onto a base material 4. The polymerization step part 20 is configured to allow a polymerizable compound, which is contained in a layer of the liquid composition obtained by applying the liquid composition onto the base material 4, to polymerize to thereby obtain a precursor 6 of an anti-pathogen structure. The heating step part 30 is configured to heat the precursor 6 of the anti-pathogen structure to obtain an anti-pathogen structure. The apparatus for producing an anti-pathogen structure 100 includes a conveying part 5 configured to convey the base material 4. The conveying part 5 is configured to convey the base material 4 in the order of the application step part 10, the polymerization step part 20, and the heating step part 30 at a previously set rate.

When the precipitation-type composition is used as the liquid composition, the polymerization step part 20 may be omitted.

—Application Step Part—

The application step part 10 includes an application device 1a, a storage container 1b, and a supply tube 1c. The application device 1a is one example of the application unit that achieves the step of applying the liquid composition onto the base material 4. The storage container 1b is configured to store the liquid composition. The supply tube 1c is configured to supply the liquid composition stored in the storage container 1b to the application device 1a.

The storage container 1b is configured to store a liquid composition 7. In the application step part 10, the liquid composition 7 is discharged from the application device 1a in a direction of the base material 4, to apply the liquid composition 7. Then, a layer of the liquid composition is formed in a thin film form.

Note that, the storage container 1b may be integrated with the apparatus for producing an anti-pathogen structure 100, but may be detachable from the apparatus for producing an anti-pathogen structure 100. The storage container 1b may be a container used to be added to the storage container integrated with the apparatus for producing an anti-pathogen structure 100 or the storage container detachable from the apparatus for producing an anti-pathogen structure 100.

The application device 1a is not particularly limited so long as it can apply the liquid composition 7. For example, it is possible to use any application device according to, for example, the spin coating method, the casting method, the microgravure coating method, the gravure coating method, the bar coating method, the roll coating method, the wire bar coating method, the dip coating method, the slit coating method, the capillary coating method, the spray coating method, the nozzle coating method, and various printing methods such as the gravure printing method, the screen printing method, the flexographic printing method, the offset printing method, the reverse printing method, and the inkjet printing method. Among them, the inkjet printing method is preferable because the liquid composition 7 can be applied to a target position of a base material. Moreover, the inkjet printing method is preferable because a uniform film thickness of the anti-pathogen structure can be achieved.

The storage container 1b or the supply tube 1c may be optionally selected so long as the liquid composition 7 can be stably stored and supplied. Materials constituting the storage container 1b or the supply tube 1c preferably have a lightproof property in a relatively short wavelength region of ultraviolet rays and visible rays. This makes it possible to prevent the liquid composition 7 from starting polymerization by natural light.

—Polymerization Step Part—

As presented in FIG. 1, the polymerization step part 20 includes a light-emitting device 2a and a polymerization-inert-gas-circulating device 2b. The light-emitting device 2a is one example of a curing unit configured to irradiate a liquid composition with active energy rays such as heat and light, to cure the liquid composition. The polymerization-inert-gas-circulating device 2b is configured to circulate polymerization inert gas. The light-emitting device 2a is configured to emit light to the layer of the liquid composition formed by the application step part 10 in the presence of the polymerization inert gas, to allow it to photopolymerize. As a result, the precursor 6 of the anti-pathogen structure is obtained.

The light-emitting device 2a is appropriately selected depending on the absorption wavelength of the photopolymerization initiator contained in the layer of the liquid composition. The light-emitting device 2a is not particularly limited so long as polymerization of a compound in the layer of the liquid composition can start and proceed. Examples thereof include high pressure mercury lamps, metal halide lamps, hot-cathode tubes, cold-cathode tubes, and light sources for ultraviolet rays such as LED. Note that, because light having a shorter wavelength generally tends to reach the deep part, it is preferable to select a light source depending on a thickness of the anti-pathogen structure to be formed.

The irradiation intensity of the light source of the light-emitting device 2a will be described. When the irradiation intensity is too strong, the polymerization proceeds drastically before phase separation occurs sufficiently. Therefore, it is difficult to obtain an anti-pathogen structure having sufficient numbers of openings and pores. Meanwhile, when the irradiation intensity is too weak, phase separation proceeds beyond a microscale. As a result, sizes of the openings and pores easily become uneven and are easily increased. In addition, the irradiation time is lengthened, and productivity tends to be decreased. Therefore, the irradiation intensity is preferably 10 mW/cm² or more but 1 W/cm² or less, more preferably 30 mW/cm² or more but 300 mW/cm² or less.

The polymerization-inert-gas-circulating device 2b decreases a concentration of oxygen having activity to polymerization contained in the air to promote the polymerization reaction of the polymerizable compound near the surface of the layer of the liquid composition without being disturbed. Therefore, the polymerization inert gas to be used is not particularly limited so long as it satisfies the aforementioned function. Examples of the polymerization inert gas include nitrogen, carbon dioxide, and argon.

Considering that its flow rate can effectively achieve the inhibition decreasing effect, a concentration of O₂ is preferably less than 20% (an environment where its oxygen concentration is lower than the oxygen concentration in the air), more preferably 0% or more but 15% or less, still more preferably 0% or more but 5% or less. The polymerization-inert-gas-circulating device 2b is preferably provided with a temperature adjustment unit configured to adjust temperatures in order to achieve stable polymerization promoting conditions.

—Heating Step Part—

As presented in FIG. 1, the heating step part 30 includes a heating device 3a that is one example of a heating unit that achieves a heating step. The heating step part 30 includes a step (solvent removing step) of heating and drying, with the heating device 3a, the solvent remaining on the precursor 6 of the anti-pathogen structure formed by the polymerization step part 20, to remove the solvent. This makes it possible to form an anti-pathogen structure. In the heating step part 30, the solvent removing step may be performed under reduced pressure.

The heating step part 30 also includes a polymerization promoting step and an initiator removing step. The polymerization promoting step is a step of heating the precursor 6 of the anti-pathogen structure using the heating device 3a to further promote the polymerization reaction performed in the polymerization step part 20. The initiator removing step is a step of heating and drying, using the heating device 3a, the photopolymerization initiator remaining in the precursor 6 of the anti-pathogen structure, to remove the initiator. Note that, these polymerization promoting step and initiator removing step may not be performed simultaneously with the solvent removing step, and may be performed before or after the solvent removing step.

The heating step part 30 further includes a step (polymerization completing step) of heating the anti-pathogen structure under reduced pressure after the solvent removing step. The heating device 3a is not particularly limited so long as it satisfies the aforementioned function. Examples of the heating device 3a include JR heaters and warm air heaters.

A heating temperature or time thereof can be appropriately selected depending on a boiling point of the solvent contained in the precursor 6 of the anti-pathogen structure or a thickness of the film to be formed.

When a precipitation-type composition is used as the liquid composition, the heating step part 30 performs heating and dries a good solvent and a poor solvent using a heating unit, to a precipitate precipitation resin dissolved or dispersed, forming an anti-pathogen structure. At this time, the drying unit that is a unit used in the step (drying step) of drying the good solvent and the poor solvent is not limited to the heating unit. For example, an air blowing unit may be used as the drying unit.

—Base Material—

As a material of the base material 4, any material can be used regardless of whether the material is transparent or opaque. Examples of the transparent base material include: glass base materials; resin film base materials such as various plastic films; and composite base materials of these base materials. Examples of the opaque base material include: metal base materials such as stainless steel; and base materials obtained by stacking the foregoing.

Regarding a shape of the base material, any shape may be used without particular limitation so long as the base material can be a base material applicable to the application step part 10 and the polymerization step part 20. For example, base materials having a curved surface shape or an uneven shape may be used.

<<Use of Anti-Pathogen Structure>>

Use of the anti-pathogen structure of the present embodiment is not particularly limited so long as the anti-pathogen structure can be exhibited. Examples of the use include such use that an anti-pathogen structure is formed on the surfaces of various base materials (e.g., resins, paper, metals, and cloths) to thereby give an anti-pathogen activity to these base materials. More specifically, the use is preferably applied to food uses and medical uses. Examples thereof include food trays, food containers, food wrap films, medical trays, medical containers, medical clothes, medical gloves, medical caps, medical masks, medical tape, antibacterial films, and antibacterial tissues.

Here, products, which include a base material and an anti-pathogen structure formed on the surface of the base material and have an anti-pathogen activity added to the base material, are referred to as an “anti-pathogen activity adduct”, in the present application.

### EXAMPLES

Examples of the present disclosure will be described hereinafter. However, the present disclosure should not be construed as being limited to these Examples.

### Preparation Example of Liquid Composition

### Preparation Example 1

Materials were mixed at the following rate to prepare a liquid composition 1.
Polymerizable compound: tricyclodecane dimethanol diacrylate (obtained from DAICEL-ALLNEX LTD.): 29.0 parts by mass
Porogen: dipropylene glycol monomethyl ether (obtained from Kanto Chemical Industry Co., Ltd.): 70.0 parts by mass
Polymerization initiator: IRGACURE 184 (obtained from BASF): 1.0 part by mass

When the liquid composition 1 was measured for a viscosity at 25 degrees Celsius using a viscometer (device name: RE-550L, obtained from Toki Sangyo Co., Ltd), it was found to have the viscosity of 30.0 mPa·s or less.

### Preparation Example 2

Materials were mixed at the following rate to prepare a liquid composition 2.
Polymerizable compound: tris(2-hydroxyethyl) isocyanurate triacrylate (obtained from ARKEMA (SARTOMER)): 29.0 parts by mass
Porogen: dipropylene glycol monomethyl ether (obtained from Kanto Chemical Industry Co., Ltd.): 70.0 parts by mass
Polymerization initiator: IRGACURE 184 (obtained from BASF): 1.0 part by mass

When the liquid composition 2 was measured for a viscosity at 25 degrees Celsius using a viscometer (device name: RE-550L, obtained from Toki Sangyo Co., Ltd), it was found to have the viscosity of 30.0 mPa·s or less.

### Comparative Preparation Example 1

Materials were mixed at the following rate to prepare a comparative liquid composition 1.
Polymerizable compound: tricyclodecane dimethanol diacrylate (obtained from DAICEL-ALLNEX LTD.): 29.0 parts by mass
Porogen: cyclohexanone (obtained from Kanto Chemical Industry Co., Ltd.): 70.0 parts by mass
Polymerization initiator: IRGACURE 184 (obtained from BASF): 1.0 part by mass

When the comparative liquid composition 1 was measured for a viscosity at 25 degrees Celsius using a viscometer (device name: RE-550L, obtained from Toki Sangyo Co., Ltd), it was found to have the viscosity of 30.0 mPa·s or less.

### Preparation Example of Anti-Pathogen Structure

### Example 1

The liquid composition 1 was loaded into an inkjet discharging apparatus equipped with a GEN5 head (obtained from Ricoh Printing Systems, Ltd.) and was discharged onto a glass plate, to form an applied region of a solid image. Immediately after that, under N₂ atmosphere, the applied region of the liquid composition 1 was irradiated with ultraviolet rays (UV) (light source: UV-LED (obtained from Phoseon, product name: FJ800), wavelength: 365 nm, irradiation intensity: 30 mW/cm², irradiation time: 20 s) to cure the applied region of the liquid composition 1. Then, a hot plate was used to heat the cured product at 120 degrees Celsius for 1 minute, to remove the porogen. As a result, an anti-pathogen structure of Example 1 was obtained. When the liquid composition 1 was discharged by an inkjet method, discharge failures such as nozzle clogging and discharge bending were not found. Therefore, the liquid composition 1 was found to have a high discharge stability.
The result obtained by observing the surface of the anti-pathogen structure of Example 1 with a scanning electron microscope (SEM) is presented in FIG. 4.

### Example 2

An anti-pathogen structure of Example 2 was obtained in the same manner as in Example 1 except that the liquid composition 1 was changed to the liquid composition 2. When the liquid composition 2 was discharged by an inkjet method, discharge failures such as nozzle clogging and discharge bending were not found. Therefore, the liquid composition 2 was found to have a high discharge stability.

### Comparative Example 1

A structure of Comparative Example 1 was obtained in the same manner as in Example 1 except that the liquid composition 1 was changed to the comparative liquid composition 1. When the comparative liquid composition 1 was discharged by an inkjet method, discharge failures such as nozzle clogging and discharge bending were not found. Therefore, the comparative liquid composition 1 was found to have a high discharge stability.

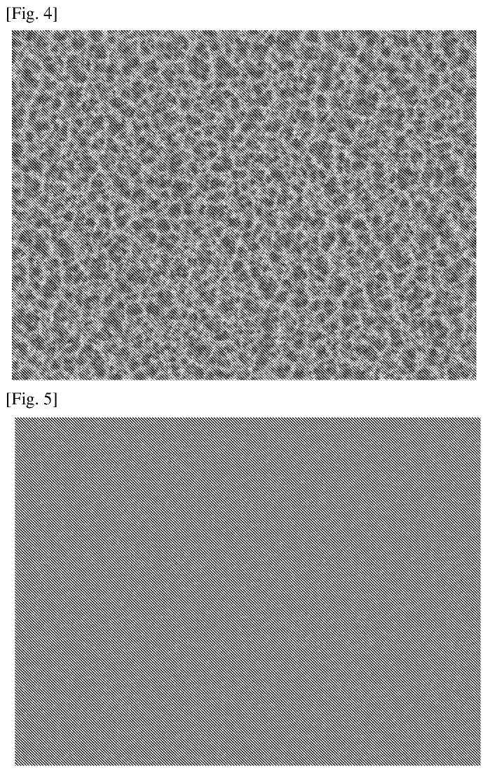

The result obtained by observing the surface of the structure of Comparative Example 1 with a scanning electron microscope (SEM) is presented in FIG. 5.

The anti-pathogen structures of Examples 1 and 2 and the structure of Comparative Example 1 obtained were evaluated for the pore diameter of the openings in the surface, the pore diameter of the inner pore, and the porosity.

<Evaluation of Pore Diameter of Openings in Surface>

The surface of the anti-pathogen structure was observed with a scanning electron microscope (SEM). As a result, openings having a pore diameter of about 1.0 micrometer were found over the whole surface of the anti-pathogen structure in Examples 1 and 2. Meanwhile, no opening was found in Comparative Example 1.

<Evaluation of Pore Diameter of Inner Pore>

A cross section of the anti-pathogen structure was prepared, and the cross section was observed with a scanning electron microscope (SEM). As a result, pores having a pore diameter of about 1.0 micrometer were found over the whole cross section of the anti-pathogen structure in Examples 1 and 2. Meanwhile, no pore was found in Comparative Example 1. It was found that the pores were coupled to each other in Examples 1 and 2, and were further coupled to the openings in the surface.

<Evaluation of Porosity>

The anti-pathogen structure was loaded with an unsaturated fatty acid (commercially available butter) and was subjected to the osmium staining. Then, the inner cross-sectional structure was cut through FIB, and the porosity of the anti-pathogen structure was measured with SEM. As a result, the porosities of Examples 1 and 2 were 30% or more. Meanwhile, the porosity of Comparative Example was less than 30%.

Next, the anti-pathogen structures of Examples 1 and 2 and the structure of Comparative Example 1 obtained were evaluated for the anti-pathogen activity (antibacterial activity).

<Evaluation of Anti-Pathogen Activity (Antibacterial Activity)>

According to the method of JIS Z 2801 (2012), the anti-pathogen activity was evaluated. Specifically, the same bacterial culture was inoculated into an unprocessed test piece (glass plate) and a sample (the anti-pathogen structures of Examples 1 and 2 and the structure of Comparative Example 1), and the viable cell count obtained after 24 hours was measured. Results are presented in the following Table 1.

TABLE 1

| | | | | Viable cell count | | |
|---|---|---|---|---|---|---|
| | | | | per 1 cm² of test piece | | |
| | At the time of | | | First | Second | Third |
| Test bacteria | measurement | Test piece | | measurement | measurement | measurement |
| Staphylococcus | Immediately | Unprocessed | Glass plate | 2.2 × 10 [symbol] | 2.4 × 10 [symbol] | 2.5 × 10 [symbol] |
| aureus | after inoculation | | | | | |
| | 35° C. | Sample | Example 1 | Less than 0.63 | Less than 0.63 | Less than 0.63 |
| | after 24 hours | | Example 2 | 7.5 | 39 | 42 |
| | | | Comparative | 1.1 × 10 [symbol] | 1.2 × 10 [symbol] | 4.3 × 10 [symbol] |
| | | | Example 1 | | | |
| | | Unprocessed | Glass plate | 1.2 × 10 [symbol] | 3.8 × 10 [symbol] | 3.9 × 10 [symbol] |
| Escherichia | Immediately | Unprocessed | Glass plate | 1.3 × 10 [symbol] | 1.5 × 10 [symbol] | 1.3 × 10 [symbol] |
| coli | after inoculation | | | | | |
| | 35° C. | Sample | Example 1 | 2.4 × 10 [symbol] | 2.6 × 10 [symbol] | 2.8 × 10 [symbol] |
| | after 24 hours | | Example 2 | 6.7 × 10 [symbol] | 1.0 × 10 [symbol] | 1.9 × 10 [symbol] |
| | | | Comparative | 6.1 × 10 [symbol] | 5.1 × 10 [symbol] | 8.1 × 10 [symbol] |
| | | | Example 1 | | | |
| | | Unprocessed | Glass plate | 8.0 × 10 [symbol] | 6.5 × 10 [symbol] | 6.4 × 10 [symbol] |

[symbol]

indicates data missing or illegible when filed

The anti-pathogen structures of Examples 1 and 2 and the structure of Comparative Example 1 obtained were evaluated for the durability and the water resistance.

<Evaluation of Durability>

First, according to the method of JIS Z 2801 (2012), the anti-pathogen structures of Examples 1 and 2 and the structure of Comparative Example 1 obtained were evaluated for the antibacterial activity value. Specifically, the same bacterial culture was each inoculated into an unprocessed test piece (test piece A) as a glass substrate, a test piece B formed on the test piece A, and a test piece C formed on the test piece A. Then, the viable cell count obtained after 24 hours was measured, and an antibacterial activity value was calculated based on the following numerical formula. The antibacterial activity value of 0.3 or more was considered as “a”, and the antibacterial activity value of less than 0.3 was considered as “b”. Results are presented in the following Table 2.

Here, the test piece C was the anti-pathogen structures of Examples 1 and 2 and the structure of Comparative Example 1.
The test piece B was a test piece prepared by using a liquid composition as described below. Specifically, the aforementioned liquid composition was obtained in the same manner as in Preparation Examples 1 and 2 and Comparative Preparation Example 1 except that porogen was not included. More specifically, the test piece B was obtained in the following manner. First, the liquid composition was coated on a glass plate to form a coated region of a solid image. Immediately after that, under N₂ atmosphere, the coated region of the liquid composition was irradiated with ultraviolet rays (UV) (light source: UV-LED (obtained from Phoseon, product name: FJ800), wavelength: 365 nm, irradiation intensity: 30 mW/cm², irradiation time: 20 s) to cure the coated region of the liquid composition. All the test pieces B prepared by using the liquid composition were each a test piece that had a plane surface structure and did not include a plurality of openings.

Antibacterial activity value=(log B−log A)−(log C−log A)
A: An average value of viable cell counts on test piece A obtained after 24 hours.
B: An average value of viable cell counts on test piece B obtained after 24 hours.
C: An average value of viable cell counts on test piece C obtained after 24 hours.

The surface of the test piece C (the anti-pathogen structures of Examples 1 and 2 and the structure of Comparative Example 1) was rubbed 10 times using a dry cotton cloth (canequim No. 3) by application of load (400 g). After the rubbing, the antibacterial activity values of the anti-pathogen structures of Examples 1 and 2 and the structure of Comparative Example 1 were determined in the above-described manner. Results are presented in the following Table 2.

<Evaluation of Water Resistance>

First, according to the method of JIS Z 2801 (2012), the anti-pathogen structures of Examples 1 and 2 and the structure of Comparative Example 1 obtained were evaluated for the antibacterial activity value. Specifically, the same bacterial culture was each inoculated into an unprocessed test piece (test piece A) as a glass substrate, a test piece B formed on the test piece A, and a test piece C formed on the test piece A. Then, the viable cell count obtained after 24 hours was measured, and an antibacterial activity value was calculated based on the following numerical formula. The antibacterial activity value of 0.3 or more was considered as “a”, and the antibacterial activity value of less than 0.3 was considered as “b”. Results are presented in the following Table 2.

Here, the test piece C was the anti-pathogen structures of Examples 1 and 2 and the structure of Comparative Example 1.
The test piece B was a test piece prepared by using a liquid composition as described below. Specifically, the aforementioned liquid composition was obtained in the same manner as in Preparation Examples 1 and 2 and Comparative Preparation Example 1 except that porogen was not included. More specifically, the test piece B was obtained in the following manner. First, the liquid composition was coated on a glass plate to form a coated region of a solid image. Immediately after that, under N₂ atmosphere, the coated region of the liquid composition was irradiated with ultraviolet rays (UV) (light source: UV-LED (obtained from Phoseon, product name: FJ800), wavelength: 365 nm, irradiation intensity: 30 mW/cm², irradiation time: 20 s) to cure the coated region of the liquid composition. All the test pieces B prepared by using the liquid composition were each a test piece that had a plane surface structure and did not include a plurality of openings.

Antibacterial activity value=(log B−log A)−(log C−log A)
A: An average value of viable cell counts on test piece A obtained after 24 hours.
B: An average value of viable cell counts on test piece B obtained after 24 hours.
C: An average value of viable cell counts on test piece C obtained after 24 hours.

The test piece C (the anti-pathogen structures of Examples 1 and 2 and the structure of Comparative Example 1) was immersed in distilled water of which temperature was maintained at 25 degrees Celsius, and was left to stand for 24 hours. Then, the resultant was further air-dried for a day. The antibacterial activity values of the anti-pathogen structures of Examples 1 and 2 and the structure of Comparative Example 1 obtained after drying were determined in the above-described manner. Results are presented in the following Table 2.

TABLE 2

| Evaluation of antibacterial activity values | |
|---|---|
| Durability | Water resistance |

| | Before | After | Before | After |
|---|---|---|---|---|
| | rubbing | rubbing | immersion | immersion |
| Example 1 | a | a | a | a |
| Example 2 | a | a | a | a |
| Comparative | b | b | b | b |
| Example 1 | | | | |

### Preparation Example of Liquid Composition

### Preparation Example 3

Materials were mixed at the following rate to prepare a comparative liquid composition 3.
Polymerizable compound: tricyclodecane dimethanol diacrylate (obtained from DAICEL-ALLNEX LTD.): 28.0 parts by mass
Porogen: ethylene glycol monobutyl ether: 70.0 parts by mass
Polymerization initiator: IRGACURE 819 (obtained from BASF): 1.0 part by mass
   When the liquid composition 3 was measured for a viscosity at 25 degrees Celsius using a viscometer (device name: RE-550L, obtained from Toki Sangyo Co., Ltd), it was found to have the viscosity of 30.0 mPa·s or less.

### Preparation Example of Anti-Pathogen Structure

### Example 3

An anti-pathogen structure of Example 3 was obtained in the same manner as in Example 1 except that the liquid composition 1 was changed to the liquid composition 3. When the liquid composition 3 was discharged by an inkjet method, discharge failures such as nozzle clogging and discharge bending were not found. Therefore, the liquid composition 3 was found to have a high discharge stability.

The anti-pathogen structures of Example 3 obtained was evaluated for the pore diameter of the openings in the surface, the pore diameter of the inner pore, and the porosity, in the same manner as in Example 1. As a result, openings having a pore diameter of from about 0.1 micrometers through 0.5 micrometers were found over the whole surface of the anti-pathogen structure. The pores having a pore diameter of from about 0.1 micrometers through 0.5 micrometers were found over the whole cross section of the anti-pathogen structure. The porosity thereof was 30% or more.

The anti-pathogen structure of Example 3 obtained was evaluated for the anti-pathogen activity (antibacterial activity).

<Evaluation of Anti-Pathogen Activity (Antibacterial Activity)>

First, according to the method of ISO 22196 (2011), the antibacterial activity value of the anti-pathogen structure of Example 3 was determined. Specifically, the same bacterial culture was each inoculated into a test piece B formed on a glass substrate and a test piece C formed on a glass substrate. Then, the viable cell count obtained after 24 hours was measured, and an antibacterial activity value was calculated based on the following numerical formula. Results are presented in Table 3.

Here, the test piece C was the anti-pathogen structure of Example 3.

The test piece B was a test piece prepared by using a liquid composition as described below. Specifically, the aforementioned liquid composition was obtained in the same manner as in Preparation Example 3 except that porogen was not included. More specifically, the test piece B was obtained in the following manner. First, the liquid composition was coated on a glass plate to form a coated region of a solid image. Immediately after that, under N₂ atmosphere, the coated region of the liquid composition was irradiated with ultraviolet rays (UV) (light source: UV-LED (obtained from Phoseon, product name: FJ800), wavelength: 365 nm, irradiation intensity: 30 mW/cm², irradiation time: 20 s) to cure the coated region of the liquid composition. The test piece B prepared by using the liquid composition was a test piece that had a plane surface structure and did not include a plurality of openings.

Antibacterial activity value=Ut−At
Ut: An average value of common logarithm values of viable cell counts on test piece B obtained after 24 hours
At: An average value of common logarithm values of viable cell counts on test piece C obtained after 24 hours

TABLE 3

| | | | Common logarithm | | |
|---|---|---|---|---|---|
| | At the time | | values of viable | | Antibacterial |
| Test bacteria | of measurement | Test piece | cell counts | | activity value |
| Staphylococcus aureus | 35° C., | B | Ut | 3.57 | 3.7 |
| | after 24 hours | C (Example 3) | At | Less than −0.20 | |
| Escherichia coli | 35° C., | B | Ut | 5.98 | 2.0 |
| | after 24 hours | C (Example 3) | At | 3.89 | |

The anti-pathogen structure of Example 3 obtained was evaluated for the durability and the water resistance.

<Evaluation of Durability>

The surface of the test piece C (the anti-pathogen structure of Example 3) was rubbed 10 times using a dry cotton cloth (canequim No. 3) by application of load (400 g). After the rubbing, the antibacterial activity value of the anti-pathogen structure of Example 3 was determined by the method according to ISO 22196 (2011) as described above. The antibacterial activity value of 0.3 or more was considered as “a”, and the antibacterial activity value of less than 0.3 was considered as “b”. Results are presented in the following Table 4.

<Evaluation of Water Resistance>

The test piece C (the anti-pathogen structure of Example 3) was immersed in distilled water of which temperature was maintained at 25 degrees Celsius, and was left to stand for 24 hours. Then, the resultant was further air-dried for a day. The antibacterial activity value of the anti-pathogen structure of Example 3 obtained after drying was determined by the method according to the ISO 22196 (2011) as described above. The antibacterial activity value of 0.3 or more was considered as “a”, and the antibacterial activity value of less than 0.3 was considered as “b”. Results are presented in the following Table 4.

TABLE 4

| Evaluation of antibacterial activity values | |
|---|---|
| Durability | Water resistance |

| | Before | After | Before | After |
|---|---|---|---|---|
| | rubbing | rubbing | immersion | immersion |
| Example 3 | a | a | a | a |

### Preparation Example of Liquid Composition

### Preparation Example 4

Materials were mixed at the following rate to prepare a liquid composition 4.
Polymerizable compound: tricyclodecane dimethanol diacrylate (obtained from DAICEL-ALLNEX LTD.): 48.0 parts by mass
Porogen: ethylene glycol monoisopropyl ether: 50.0 parts by mass
Polymerization initiator: IRGACURE 819 (obtained from BASF): 1.0 part by mass

When the liquid composition 4 was measured for a viscosity at 25 degrees Celsius using a viscometer (device name: RE-550L, obtained from Toki Sangyo Co., Ltd), it was found to have the viscosity of 30.0 mPa·s or less.

### Preparation Example of Anti-Pathogen Structure

### Example 4

The liquid composition 4 was loaded into an inkjet discharging apparatus equipped with a GEN5 head (obtained from Ricoh Printing Systems, Ltd.) and was discharged onto a glass plate, to form an applied region of a solid image. Immediately after that, under N₂ atmosphere, the applied region of the liquid composition 4 was irradiated with ultraviolet rays (UV) (light source: UV-LED (obtained from Phoseon, product name: FJ800), wavelength: 365 nm, irradiation intensity: 400 mW/cm², irradiation time: 20 s) to cure the applied region of the liquid composition 4. Then, a hot plate was used to heat the cured product at 120 degrees Celsius for 1 minute, to remove the porogen. As a result, an anti-pathogen structure of Example 4 was obtained. When the liquid composition 4 was discharged by an inkjet method, discharge failures such as nozzle clogging and discharge bending were not found. Therefore, the liquid composition 4 was found to have a high discharge stability.

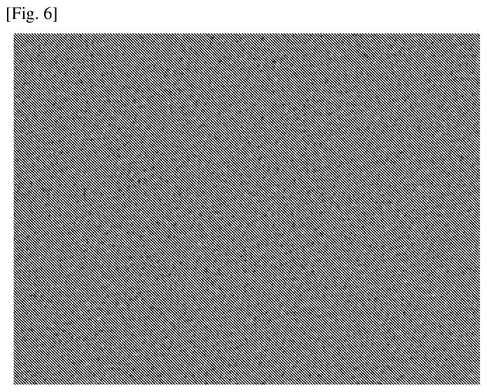

The result obtained by observing the surface of the anti-pathogen structure of Example 4 with a scanning electron microscope (SEM) is presented in FIG. 6.

The anti-pathogen structure of Example 4 obtained was evaluated for the pore diameter of the openings in the surface, the pore diameter of the inner pore, and the porosity in the same manner as in Example 1.

As a result, openings having a pore diameter of about 0.05 micrometers were found over the whole surface of the anti-pathogen structure. The pores having a pore diameter of about 0.05 micrometers were found over the whole cross section of the anti-pathogen structure. The porosity thereof was 30% or more.

The anti-pathogen structures of Example 1 and Example 4 obtained were evaluated for an anti-pathogen activity (antiviral activity).

<Evaluation of Anti-Pathogen Activity (Antiviral Activity)>

According to the method of ISO 21702 (2019), the antiviral activity values of the anti-pathogen structures of Example 1 and Example 4 were determined. Specifically, the same viral culture was each inoculated into a test piece Y formed on a glass plate and a test piece X formed on a glass plate. A viral infectivity titer (PFU/cm²) thereof obtained after 24 hours was measured, to calculate an antiviral activity value based on the following numerical formula. Results are presented in the following Table 5.

Here, the test piece X was the anti-pathogen structure of Example 1 or Example 4.

The test piece Y was a test piece prepared by using a liquid composition as described below. Specifically, the aforementioned liquid composition was obtained in the same manner as in Preparation Example 1 or Preparation Example 4 except that porogen was not included. More specifically, the test piece Y was obtained in the following manner. First, the liquid composition was coated on a glass plate to form a coated region of a solid image. Immediately after that, under N₂ atmosphere, the coated region of the liquid composition was irradiated with ultraviolet rays (UV) (light source: UV-LED (obtained from Phoseon, product name: FJ800), wavelength: 365 nm, irradiation intensity: 30 mW/cm², irradiation time: 20 s) to cure the coated region of the liquid composition. The test piece Y prepared by using the liquid composition was a test piece that had a plane surface structure and did not include a plurality of openings.

Antiviral activity value=Ut−At
Ut: An average value of common logarithm values of viral infectivity titers on test piece Y obtained after 24 hours
At: An average value of common logarithm values of viral infectivity titers on test piece X obtained after 24 hours

TABLE 5

| | | | Common logarithm values | | Antiviral |
|---|---|---|---|---|---|
| | At the time | | of viral infectivity titers | | activity |
| Test bacteria | of measursement | Test piece | (PFU/cm²) | | value |
| Influenza Virus | 35° C., | Y | Ut | 5.11 | 0.7 |
| | after 24 hours | X (Example 1) | At | 4.34 | |
| | | Y | Ut | 5.11 | 4.0 |
| | | X (Example 4) | At | 1.06 | |

The anti-pathogen structures of Example 1 and Example 4 obtained were evaluated for the durability and the water resistance.

<Evaluation of Durability>

The surface of the test piece X (the anti-pathogen structures of Example 1 and Example 4) was rubbed 10 times using a dry cotton cloth (canequim No. 3) by application of load (400 g). After the rubbing, the antiviral activity values of the anti-pathogen structure of Example 1 and Example 4 were determined by the method according to ISO 21702 (2019) as described above. The antiviral activity value of 0.2 or more was considered as “a”, and the antiviral activity value of less than 0.2 was considered as “b”. Results are presented in the following Table 6.

<Evaluation of Water Resistance>

The test piece X (the anti-pathogen structures of Example 1 and Example 4) was immersed in distilled water of which temperature was maintained at 25 degrees Celsius, and was left to stand for 24 hours. Then, the resultant was further air-dried for a day. The antiviral activity values of the anti-pathogen structures of Example 1 and Example 4 obtained after drying were determined by the method according to ISO 21702 (2019) as described above. The antiviral activity value of 0.2 or more was considered as “a”, and the antiviral activity value of less than 0.2 was considered as “b”. Results are presented in the following Table 6.

TABLE 6

| Evaluation of antiviral activity values | |
|---|---|
| Durability | Water resistance |

| | Before | After | Before | After |
|---|---|---|---|---|
| | rubbing | rubbing | immersion | immersion |
| Example 1 | a | a | a | a |
| Example 4 | a | a | a | a |

### Preparation Example of Liquid Composition

### Preparation Example 5

Materials were mixed at the following rate to prepare a liquid composition 5.
Polymerizable compound: tricyclodecane dimethanol diacrylate (obtained from DAICEL-ALLNEX LTD.): 29.0 parts by mass
Porogen: dipropylene glycol monomethyl ether (obtained from Kanto Chemical Industry Co., Ltd.): 65.0 parts by mass
Polymerization initiator: IRGACURE 184 (obtained from BASF): 1.0 part by mass
Polyvinyl butyral resin (obtained from Kuraray Co., Ltd., Mowital B20H): 5.0 parts by mass

When the liquid composition 5 was measured for a viscosity at 25 degrees Celsius using a viscometer (device name: RE-550L, obtained from Toki Sangyo Co., Ltd), it was found to have the viscosity of 100.0 mPa·s or less.

### Preparation Example of Anti-Pathogen Structure

### Example 5

The liquid composition 5 was coated onto a glass plate, to form an applied region of a solid image. Immediately after that, under N₂ atmosphere, the applied region of the liquid composition 5 was irradiated with ultraviolet rays (UV) (light source: UV-LED (obtained from Phoseon, product name: FJ800), wavelength: 365 nm, irradiation intensity: 30 mW/cm², irradiation time: 20 s) to cure the applied region of the liquid composition 5. Then, a hot plate was used to heat the cured product at 120 degrees Celsius for 1 minute, to remove the porogen. As a result, an anti-pathogen structure of Example 5 was obtained.
The result obtained by observing the surface of the anti-pathogen structure of Example 5 with a scanning electron microscope (SEM) is presented in FIG. 3 as described above.

The anti-pathogen structure of Example 5 obtained was evaluated for the pore diameter of the openings in the surface, the pore diameter of the inner pore, and the porosity in the same manner as in Example 1.

As a result, openings having a pore diameter of about 0.5 micrometers were found over the whole surface of the anti-pathogen structure. The pores having a pore diameter of about 0.5 micrometers were found over the whole cross section of the anti-pathogen structure. The porosity thereof was 15% or more.

The anti-pathogen structure of Example 5 obtained was evaluated for the pencil hardness.

<Evaluation of Pencil Hardness>

According to the method of ISO 15184, the hardness of the surface, on which the surface structure of the anti-pathogen structure (resin structure) of Example 5 was formed, was determined. This measurement was performed by application of load (750 g) using a pencil hardness tester (obtained from Toyo Seiki Seisaku-sho, Ltd.).

As a result, the pencil hardness of the anti-pathogen structure of Example 5 was F.

The anti-pathogen structure of Example 5 obtained was evaluated for the anti-pathogen activity (antibacterial activity) according to the method of ISO 22196 (2011) similarly to Example 3.

As a result, the antibacterial activity value of the anti-pathogen structure of Example 5 was 0.3 or more.

The anti-pathogen structure of Example 5 obtained was evaluated for the durability in the same manner as in Example 3.

As a result, the antibacterial activity value of the anti-pathogen structure of Example 5 obtained after rubbing was 0.3 or more.

The anti-pathogen structure of Example 5 obtained was evaluated for the water resistance in the same manner as in Example 3.

The antibacterial activity value, which was obtained after immersing the anti-pathogen structure of Example 5 in distilled water followed by drying, was 0.3 or more.

### Preparation Example of Liquid Composition

### Preparation Example 6

Materials were mixed at the following rate to prepare a liquid composition 6.
Precipitation resin: polylactic acid-glycolic acid copolymer (PLGA7520, obtained from FUJIFILM Wako Pure Chemical Corporation): 10.0 parts by mass
Good solvent: acetone: 67.5 parts by mass (obtained from FUJIFILM Wako Pure Chemical Corporation)
Poor solvent: ethanol: 22.5 parts by mass (obtained from FUJIFILM Wako Pure Chemical Corporation)

When the liquid composition 6 was measured for a viscosity at 25 degrees Celsius using a viscometer (device name: RE-550L, obtained from Toki Sangyo Co., Ltd), it was found to have the viscosity of 30.0 mPa·s or less.

### Preparation Example of Liquid Composition

### Preparation Example 7

Materials were mixed at the following rate to prepare a liquid composition 7.
Precipitation resin: polylactic acid (RESOMER R 203H, obtained from Sigma-Aldrich): 15.0 parts by mass
Good solvent: methyl ethyl ketone: 45.0 parts by mass (obtained from FUJIFILM Wako Pure Chemical Corporation)
Poor solvent: methanol: 45.0 parts by mass (obtained from FUJIFILM Wako Pure Chemical Corporation)

When the liquid composition 7 was measured for a viscosity at 25 degrees Celsius using a viscometer (device name: RE-550L, obtained from Toki Sangyo Co., Ltd), it was found to have the viscosity of 30.0 mPa·s or less.

### Preparation Example of Anti-Pathogen Structure

### Example 1

Each of the liquid compositions 6 and 7 was loaded into an inkjet discharging apparatus equipped with a GEN5 head (obtained from Ricoh Printing Systems, Ltd.) and was discharged onto a glass plate, to form an applied region of a solid image. Immediately after that, the glass plate was placed in a vacuum dryer temperature of which was set to 25 degrees Celsius, and was dried for 6 hours, to remove the good solvent and the poor solvent. As a result, anti-pathogen structures of Examples 6 and 7 were obtained. When the liquid compositions 6 and 7 were discharged by an inkjet method, discharge failures such as nozzle clogging and discharge bending were not found. Therefore, the liquid compositions 6 and 7 were found to have a high discharge stability.

The anti-pathogen structures of Examples 6 and 7 obtained were evaluated for the pore diameter of the openings in the surface, the pore diameter of the inner pore, and the porosity in the same manner as in Example 1.

As a result, openings having a pore diameter of about 0.5 micrometers were found over the whole surface of the anti-pathogen structure. The pores having a pore diameter of about 0.5 micrometers were found over the whole cross section of the anti-pathogen structure. The porosity thereof was 15% or more.

The anti-pathogen structures of Examples 6 and 7 obtained were evaluated for the anti-pathogen activity (antibacterial activity) according to the method of ISO 22196 (2011), similarly to Example 3.

As a result, the antibacterial activity values of the anti-pathogen structures of Examples 6 and 7 were 0.3 or more.

The anti-pathogen structures of Examples 6 and 7 obtained were evaluated for the durability in the same manner as in Example 3.

As a result, the antibacterial activity values of the anti-pathogen structures of Examples 6 and 7 obtained after rubbing were 0.3 or more.

The anti-pathogen structures of Examples 6 and 7 obtained were evaluated for the water resistance in the same manner as in Example 3.

The antibacterial activity values, which were obtained after immersing the anti-pathogen structures of Examples 6 and 7 in distilled water followed by drying, were 0.3 or more.

### REFERENCE SIGNS LIST

1a: application device
1b: container
1c: supply tube
2a: light-emitting device
2b: polymerization-inert-gas-circulating device
3a: heating device
4: base material
5: conveying part
6: precursor of anti-pathogen structure
7: liquid composition
10: application step part
20: polymerization step part
30: heating step part
100: production apparatus

1: An anti-pathogen structure, comprising:
a resin structure having a plurality of openings in a surface of the resin structure,
wherein the resin structure has an antimicrobial activity or an antiviral activity.

2: The anti-pathogen structure according to claim 1, wherein the resin structure has a porous structure, the porous structure having a co-continuous structure in which a plurality of pores are continuously coupled to each other, and the plurality of openings are each independently coupled to some of the plurality of pores constituting the co-continuous structure.

3: The anti-pathogen structure according to claim 1, wherein the resin structure has the antimicrobial activity or the antiviral activity even after the anti-pathogen structure is immersed in water of 25 degrees Celsius for 24 hours.

4: The anti-pathogen structure according to claim 1, wherein the resin structure has the antimicrobial activity, and a pore diameter of the openings is 10 micrometers or less.

5: The anti-pathogen structure according to claim 1, wherein the resin structure has the antiviral activity, and a pore diameter of the openings is 0.1 micrometers or less.

6: The anti-pathogen structure according to claim 1, wherein having the antimicrobial activity means that an antibacterial activity value of the anti-pathogen structure is 0.3 or more, the antibacterial activity value being evaluated according to a method described in JIS Z 2801 (2012) or ISO 22196 (2011).

7: The anti-pathogen structure according to claim 1, wherein having the antiviral activity means that an antiviral activity value of the anti-pathogen structure is 0.2 or more, the antiviral activity value being evaluated according to a method described in ISO 21702 (2019).

8: The anti-pathogen structure according to claim 1, wherein a porosity of the resin structure is 10% or more.

9: The anti-pathogen structure according to claim 1, wherein the anti-pathogen structure is substantially free of an antimicrobial agent and an antiviral agent.

10: The anti-pathogen structure according to claim 1, wherein the resin structure includes a skeleton that shapes the plurality of openings, and the skeleton has such a shape that a plurality of particles are coupled to each other.

11: The anti-pathogen structure according to claim 1, wherein the resin structure includes a skeleton that shapes the plurality of openings, and the skeleton has a substantially plane shape.

12: The anti-pathogen structure according to claim 1, wherein a pencil hardness of the surface of the resin structure is B or harder, the pencil hardness being evaluated according to a method described in ISO 15184.

13: A method for producing an anti-pathogen structure that has a resin structure, the resin structure having a plurality of openings in a surface of the resin structure, the method comprising:
applying a liquid composition including a polymerizable compound and a solvent; and
allowing the polymerizable compound to polymerize to form the resin structure,
wherein the resin structure has an antimicrobial activity or an antiviral activity.

14: The method for producing an anti-pathogen structure according to claim 13, wherein the applying is discharging the liquid composition.

15: The method for producing an anti-pathogen structure according to claim 13, wherein a viscosity of the liquid composition at 25 degrees Celsius is 1 mPa·s or more but 200 mPa·s or less.

16: An apparatus for producing an anti-pathogen structure that has a resin structure, the resin structure having a plurality of openings in a surface of the resin structure, the apparatus comprising:
an application unit configured to apply a liquid composition including a polymerizable compound and a solvent; and
a polymerization unit configured to allow the polymerizable compound to polymerize to form the resin structure,
wherein the resin structure has an antimicrobial activity or an antiviral activity.

17: The apparatus for producing an anti-pathogen structure according to claim 16, wherein the application unit is a unit configured to discharge the liquid composition.

18: The apparatus for producing an anti-pathogen structure according to claim 16, wherein a viscosity of the liquid composition at 25 degrees Celsius is 1 mPa·s or more but 200 mPa·s or less.

19-27. (canceled)
